SEQUENCE LISTING (14 sequences)

SEQ ID NO: 1 - DNA, 6084 bp, Mus musculus
atgtggacccccacggaggaggagaaatacggcgtagtgatatgcagctttcgaggatctgtccctcaaggcctggtcttagaaataggagaaacagttcagattcttgaaaaatgtgaaggttggtacagaggagtttcaacaaagaagcctaatgtaaaggggctctttcctgcaaattatattcatttgaaaaaggcaattgtcagtaacagggggcagtatgaaactgtggttccgctggaagattctattgtgactgaagttacaacaactctacaagagtgggccagtctgtggaaacaactatatgtgaaacacaaagtagatcttttctacaaactccgccatgtgatgaatgaacttattgacctgcgaaggcagctactgtctggtcacctgacacaggatcaggtgcgggaggttaagcggcacatcaccgtgcgtctggactggggtaatgagcatttgggcctggacttggtgcctcggaaggactttgaagtagtggattcagaccagattagcgtctcagatctctataaaatgcatttatctagccggcagagtgtacagcagagcacatcccaagtagatacaatgcgcccaaggcatggggaaacctgtcgaatgccggtaccatatcatttcttcttcagcctgaagagtttcacctataataccattggtgaagatagtgatgtcttcttttctctgtatgacatgagagaaggcaagcagatcagtgaacggtttctggtgagactgaacaagaatggtgggccaaggaacccagagaagatagaacgaatgtgtgccctttttacagatctgagcagcaaagatatgaagagagatttatacatagttgcccatgtgatccgaataggccggatgctcctgaatgactccaaaaagggtcctgctcacctgcactacaggcgaccatatggctgtgctgttctaagcatattggatgttctacagtcactaacagaattaaaagaagaaaaggattttgttcttaaggtgtacacatgtaacaacgagagtgaatggacccagattcatgagaacatcatccgaaagtcaagtaccaagtactctgcccccagtgccagccatggtcttattatttcccttcagctttttcgtggtgacatggaacagatccggagagaaaatcccatgatatttaatagaggattagcaattacaagaaaattgggatttcctgatgttattatgccaggtgatatccgcaatgatttgtacctaacactggagaagggagactttgaaagagggggaaagagtgtacaaaagaatattgaagtaaccatgtatgtactttatgcagatggagaaatcttgaaggactgtatcagtttgggttcaggagagccaaataggagttcctatcactcctttgttctctaccacagtaatagtcctcgctggggagaaattatcaaattacctattcccattgacagattccggggctcccatctccgctttgagttcagacattgctccacaaaggacaaaggggaaaagaaactctttggctttgcgttctctccgctgatgcgtgatgatggcaccacactctcagatgatattcatgagctttatgtgtataagtgtgatgagaatagcacatttaacaaccatgccctctacctgggcctgccctgctgcaaagaagactacaatggctgccctaatatcccctccagcctcatcttccagcgcagcaaagagtctttcttcatttctacacaactctcttctaccaaactcacacaaaatgtggacctccttgctctgctgaaatggaaggccttcccagaccggatcatggacatattagggcggttgcggcatgtcagtggggaagaaattgttaagtttctgcaggatatcttagatacactctttgtgattttggatgacaatacagaaaaatacggccttttggtttttcagtctctggttttcattatcaacctgcttcgagacataaaatacttccacttccgacctgtaatggacacatatatccagaagcactttgctggagccctggcatataaggagctcatccgctgtttgaagtggtacatggactgctcagcagaactgattcgacaggaccacattcaggaagccatgcgggccttggagtaccttttcaagttcattgtgcagtcacgaatcctgtattcgagagccacatgtgggatggaagaggagcagttccggtctagcatccaggaacttttccagtccatccggtttgtgctgagtctggacagtaggaactcagaaacactcctcttcactcaggctgcactactcaactccttccccaccatcttcgacgagctgctacagatgtttacggtgcaagaggtagcagagtttgtgagaggcacactgggaagcatgcctagcactgtgcacatcgggcagtcaatggatgtggtgaagctgcagtccatcgccaggacagtggatagtcgcctattttcattttcagaatcccgccgcatcctacttcctgtggttcttcatcacattcatcttcacctgaggcaacagaaagagttgctaatctgctcagggattctgggcagcatcttctccatcgtcaagaccagctctctggaggcagatgtcatggaggaggtagagatgatggtggaaagcctcctggatgttctcctacagactctcctcaccatcatgagtaaatcacatgctcaggaggcggtaagagggcactgcccagtgacagccgagatcactggtgaatatgtgtcctgcctcctctcattgctccgtcagatgtgtgacacccatttccaacacctcttagacaactttcagagtaaagatgaactaaaggaatttctgctgaagattttctgtgtgttcagaaacctaatgaagatgagtgtcttccctcgagactggatggtgatgagactgttgacaagcaacattatagtcactactgtccagtacctgtcctctgcactgcataagaatttcacagaaacagactttgacttcaaggtgtggaattcttattttagcctggcagttctgttcataaaccagccaagccttcagctagaaatcattacttcagccaagaggaagaagattctagataagtatggggacatgcgtgtgatgatggcatatgaactgttcagcatgtggcagaatctgggtgaacataagatccactttattcctggaatgattggtcctttcctgggtgtgacactggtcccacagccagaagtgcggaatatcatgattcctatctttcatgacatgatggactgggaacaaaggaaaaatggcaacttcaagcaggtggaggctgagctgattgacaagctggacagcatggtgtcagaggggaaaggtgatgagagctacagggaactcttcggcctgctaacccagctgtttgggccctaccccagccttctggaaaaggttgaacaggaaacatggcgtgagactggcatttcctttgttacctcagtcacgcgcctcatggaacgccttcttgactacagggactgcatgaagggagaagaaactgagaacaagaaggttggctgcactgttaacctgatgaacttttacaaatctgagattaacaaagaggaaatgtatatccgatacatccataaactttgtgacatgcacttacaggctgagaactacacagaggctgcattcaccctgcttctttactgcgagctgctacaatgggaagaccggccattacgggagttcctacactacccatcccaaactgagtggcagcggaaagagggactgtgtagaaagatcatccactacttcaacaaaggcaagagctgggagtttggaattcctttatgtagagagctggcatgccagtacgagagcctctatgactaccagagcctcagctggatccggaaaatggaggccagctactatgacaacatcatagagcagcaacgcctggaacctgagttcttccgggttggtttttatggcaggaagtttcctttcttccttcggaacaaagaatatgtgtgccgtggtcatgactacgagaggctagaagccttccaacagaggatgctgagcgagttcccacaggccgttgctatgcagcaccccaatcatcctgatgatgccatcctacagtgtgatgctcagtatttacagatctatgcagtgacacctattccagattacgtggatgttctacaaatggatagagtacctgatcgtgtcaagagcttctatcgtgtcaataatgtgaggaagttccggtatgatagacctttccacaaaggccccaaggacaaagacaatgaattcaagagcctgtggattgagcgcaccacgctgacactgacccacagtttgcctggcatctctcggtggtttgaagtggaaagaagggaactggtggaggtgagccctctggagaatgcaatccaggtagttgagaataagaaccaggaactgcgagctctgatcagccagtatcagcacaagcaggtacatggcaacatcaatctgctcagcatgtgcctgaacggagtcattgacgctgctgtcaatggaggcattgcacgctaccaggaggccttttttgataaagattatatcaccaagcacccaggagatgctgagaagatctcccagctcaaagaactaatgcaggaacaggtccatgttcttggagttgggctggcggttcatgagaagtttgtgcatccggaaatgcggcctctgcataagaagctgattgatcagttccagatgatgagggccagtctgtaccatgagtttccaggtttggataagctaagtcctgcatgttcaggcaccagcaccccacggggaaatgttctggcatcccatagccccatgagccctgagaacatcaagatgacccacaggcacagccccatgaacttgatgggcacaggccgccattcctcatcctctctctcctcacatgcatctagtgaagcaggaaacatgatgatgatgggtgacaactctatgggtgaagctcctgaagacctctaccaccacatgcagcttgcatatcacaaccccaggtaccagggctcagtcaccaacgtctctgttctgtcctcgtcccaggcaagcccttcttcctccagcctgagttctacacattcagctccgtcccagatgatcacctctgccccttccagcacccgaggctctccctctctgccagataagtaccgacatgcccgggaaatgatgttgctgttgcctacacatcgggaccgcccaagcagtgccatgtatccagcagccatcctagagaatggacagccaccaaacttccagcgggccctgttccagcaagtagttggagcctgtaaaccctgcagtgaccccaatctgtctatggctgaaaaaggtcattactccctacacttcgacgccttccaccaccctctgggtgatacccccccggccctccctgcccggacactgcgcaagtctcctctccaccccatcccagcttcccctacaagcccccaatcgggcctggatggcagcaactccacgctgtctggcagcgccagtagcggtgtatcctccttaagtgagagtaactttgggcattcctcagaggccccacctcgaacagacaccatggactccatgccaagccaggcctggaatggcgatgaaggccttgagccgccctacctccctgtccactacagcctctctgaatctgctgtccttgatgccatcaagtcccagccatgcaggagccactcagccccagggtgtgtcctccctcaggaccccatggacccacctgcgctgccacccaagccctatcatccccgcctgccagccctagagcatgatgagggtatgctattgcgggaggaggctgagaggccaaggggcctgcaccgcaaggcctccctgcctcctggaagtgtcaaggaggaacaggcccgcctggcctgggagcacggccgaggggaacagtga

SEQ ID NO: 2 - protein, 2027 aa, Mus musculus
MWTPTEEEKYGVVICSFRGSVPQGLVLEIGETVQILEKCEGWYRGVSTKKPNVKGLFPANYIHLKKAIVSNRGQYETVVPLEDSIVTEVTTTLQEWASLWKQLYVKHKVDLFYKLRHVMNELIDLRRQLLSGHLTQDQVREVKRHITVRLDWGNEHLGLDLVPRKDFEVVDSDQISVSDLYKMHLSSRQSVQQSTSQVDTMRPRHGETCRMPVPYHFFFSLKSFTYNTIGEDSDVFFSLYDMREGKQISERFLVRLNKNGGPRNPEKIERMCALFTDLSSKDMKRDLYIVAHVIRIGRMLLNDSKKGPAHLHYRRPYGCAVLSILDVLQSLTELKEEKDFVLKVYTCNNESEWTQIHENIIRKSSTKYSAPSASHGLIISLQLFRGDMEQIRRENPMIFNRGLAITRKLGFPDVIMPGDIRNDLYLTLEKGDFERGGKSVQKNIEVTMYVLYADGEILKDCISLGSGEPNRSSYHSFVLYHSNSPRWGEIIKLPIPIDRFRGSHLRFEFRHCSTKDKGEKKLFGFAFSPLMRDDGTTLSDDIHELYVYKCDENSTFNNHALYLGLPCCKEDYNGCPNIPSSLIFQRSKESFFISTQLSSTKLTQNVDLLALLKWKAFPDRIMDILGRLRHVSGEEIVKFLQDILDTLFVILDDNTEKYGLLVFQSLVFIINLLRDIKYFHFRPVMDTYIQKHFAGALAYKELIRCLKWYMDCSAELIRQDHIQEAMRALEYLFKFIVQSRILYSRATCGMEEEQFRSSIQELFQSIRFVLSLDSRNSETLLFTQAALLNSFPTIFDELLQMFTVQEVAEFVRGTLGSMPSTVHIGQSMDVVKLQSIARTVDSRLFSFSESRRILLPVVLHHIHLHLRQQKELLICSGILGSIFSIVKTSSLEADVMEEVEMMVESLLDVLLQTLLTIMSKSHAQEAVRGHCPVTAEITGEYVSCLLSLLRQMCDTHFQHLLDNFQSKDELKEFLLKIFCVFRNLMKMSVFPRDWMVMRLLTSNIIVTTVQYLSSALHKNFTETDFDFKVWNSYFSLAVLFINQPSLQLEIITSAKRKKILDKYGDMRVMMAYELFSMWQNLGEHKIHFIPGMIGPFLGVTLVPQPEVRNIMIPIFHDMMDWEQRKNGNFKQVEAELIDKLDSMVSEGKGDESYRELFGLLTQLFGPYPSLLEKVEQETWRETGISFVTSVTRLMERLLDYRDCMKGEETENKKVGCTVNLMNFYKSEINKEEMYIRYIHKLCDMHLQAENYTEAAFTLLLYCELLQWEDRPLREFLHYPSQTEWQRKEGLCRKIIHYFNKGKSWEFGIPLCRELACQYESLYDYQSLSWIRKMEASYYDNIIEQQRLEPEFFRVGFYGRKFPFFLRNKEYVCRGHDYERLEAFQQRMLSEFPQAVAMQHPNHPDDAILQCDAQYLQIYAVTPIPDYVDVLQMDRVPDRVKSFYRVNNVRKFRYDRPFHKGPKDKDNEFKSLWIERTTLTLTHSLPGISRWFEVERRELVEVSPLENAIQVVENKNQELRALISQYQHKQVHGNINLLSMCLNGVIDAAVNGGIARYQEAFFDKDYITKHPGDAEKISQLKELMQEQVHVLGVGLAVHEKFVHPEMRPLHKKLIDQFQMMRASLYHEFPGLDKLSPACSGTSTPRGNVLASHSPMSPENIKMTHRHSPMNLMGTGRHSSSSLSSHASSEAGNMMMMGDNSMGEAPEDLYHHMQLAYHNPRYQGSVTNVSVLSSSQASPSSSSLSSTHSAPSQMITSAPSSTRGSPSLPDKYRHAREMMLLLPTHRDRPSSAMYPAAILENGQPPNFQRALFQQVVGACKPCSDPNLSMAEKGHYSLHFDAFHHPLGDTPPALPARTLRKSPLHPIPASPTSPQSGLDGSNSTLSGSASSGVSSLSESNFGHSSEAPPRTDTMDSMPSQAWNGDEGLEPPYLPVHYSLSESAVLDAIKSQPCRSHSAPGCVLPQDPMDPPALPPKPYHPRLPALEHDEGMLLREEAERPRGLHRKASLPPGSVKEEQARLAWEHGRGEQ

SEQ ID NO: 3 - protein, 22 aa, Homo sapiens
EERGVKLGLGDFIFYSVLVGKA

SEQ ID NO: 4 - protein, 8 aa, Artificial Sequence
  A consensus sequence
PPXLPXKR

SEQ ID NO: 5 - protein, 1865 aa, Homo sapiens
MTRWVPTKREEKYGVAFYNYDARGADELSLQIGDTVHILETYEGWYRGYTLRKKSKKGIFPASYIHLKEAIVEGKGQHETVIPGDLPLIQEVTTTLREWSTIWRQLYVQDNREMFRSVRHMIYDLIEWRSQILSGTLPQDELKELKKKVTAKIDYGNRILDLDLVVRDEDGNILDPELTSTISLFRAHEIASKQVEERLQEEKSQKQNIDINRQAKFAATPSLALFVNLKNVVCKIGEDAEVLMSLYDPVESKFISENYLVRWSSSGLPKDIDRLHNLRAVFTDLGSKDLKREKISFVCQIVRVGRMELRDNNTRKLTSGLRRPFGVAVMDVTDIINGKVDDEDKQHFIPFQPVAGENDFLQTVINKVIAAKEVNHKGQGLWVTLKLLPGDIHQIRKEFPHLVDRTTAVARKTGFPEIIMPGDVRNDIYVTLVQGDFDKGSKTTAKNVEVTVSVYDEDGKRLEHVIFPGAGDEAISEYKSVIYYQVKQPRWFETVKVAIPIEDVNRSHLRFTFRHRSSQDSKDKSEKIFALAFVKLMRYDGTTLRDGEHDLIVYKAEAKKLEDAATYLSLPSTKAELEEKGHSATGKSMQSLGSCTISKDSFQISTLVCSTKLTQNVDLLGLLKWRSNTSLLQQNLRQLMKVDGGEVVKFLQDTLDALFNIMMENSESETFDTLVFDALVFIIGLIADRKFQHFNPVLETYIKKHFSATLAYTKLTKVLKNYVDGAEKPGVNEQLYKAMKALESIFKFIVRSRILFNQLYENKGEADFVESLLQLFRSINDMMSSMSDQTVRVKGAALKYLPTIVNDVKLVFDPKELSKMFTEFILNVPMGLLTIQKLYCLIEIVHSDLFTQHDCREILLPMMTDQLKYHLERQEDLEACCQLLSHILEVLYRKDVGPTQRHVQIIMEKLLRTVNRTVISMGRDSELIGNFVACMTAILRQMEDYHYAHLIKTFGKMRTDVVDFLMETFIMFKNLIGKNVYPFDWVIMNMVQNKVFLRAINQYADMLNKKFLDQANFELQLWNNYFHLAVAFLTQESLQLENFSSAKRAKILNKYGDMRRQIGFEIRDMWYNLGQHKIKFIPEMVGPILEMTLIPETELRKATIPIFFDMMQCEFHSTRSFQMFENEIITKLDHEVEGGRGDEQYKVLFDKILLEHCRKHKYLAKTGETFVKLVVRLMERLLDYRTIMHDENKENRMSCTVNVLNFYKEIEREEMYIRYLYKLCDLHKECDNYTEAAYTLLLHAKLLKWSEDVCVAHLTQRDGYQATTQGQLKEQLYQEIIHYFDKGKMWEEAIALGKELAEQYENEMFDYEQLSELLKKQAQFYENIVKVIRPKPDYFAVGYYGQGFPTFLRGKVFIYRGKEYERREDFEARLLTQFPNAEKMKTTSPPGDDIKNSPGQYIQCFTVKPKLDLPPKFHRPVSEQIVSFYRVNEVQRFEYSRPIRKGEKNPDNEFANMWIERTIYTTAYKLPGILRWFEVKSVFMVEISPLENAIETMQLTNDKINSMVQQHLDDPSLPINPLSMLLNGIVDPAVMGGFANYEKAFFTDRYLQEHPEAHEKIEKLKDLIAWQIPFLAEGIRIHGDKVTEALRPFHERMEACFKQLKEKVEKEYGVRIMPSSLDDRRGSRPRSMVRSFTMPSSSRPLSVASVSSLSSDSTPSRPGSDGFALEPLLPKKMHSRSQDKLDKDDLEKEKKDKKKEKRNSKHQEIFEKEFKPTDISLQQSEAVILSETISPLRPQRPKSQVMNVIGSERRFSVSPSSPSSQQTPPPVTPRAKLSFSMQSSLELNGMTGADVADVPPPLPLKGSVADYGNLMENQDLLGSPTPPPPPPHQRHLPPPLPSKTPPPPPPKTTRKQTSVDSGIVQ

SEQ ID NO: 6 - protein, 9 aa, Artificial Sequence
  A TAG epitope
YPYDVPDYA

SEQ ID NO: 7 - protein, 8 aa, Artificial Sequence
  A FLAG epitope
DYKDDDDI

SEQ ID NO: 8 - protein, 57 aa, Mus musculus
KYGVVICSFRGSVPQGLVLEIGETVQILEKCEGWYRGVSTKKPNVKGLFPANYIHLK

SEQ ID NO: 9 - protein, 57 aa, Homo sapiens
KYGVAFYNYDARGADELSLQIGDTVHILETYEGWYRGYTLRKKSKKGIFPASYIHLK

SEQ ID NO: 10 - protein, 55 aa, Homo sapiens
RTVKALYDYKAKRSDELSFCRGALIHNVSKEPGGWWKGDYGTRIQQYFPSNYVEI

SEQ ID NO: 11 - protein, 57 aa, Homo sapiens
TTFVALYDYESRTETDLSFKKGERLQIVNNTEGDWWLAHSLSTGQTGYIPSNYVAPS

SEQ ID NO: 12 - protein, 63 aa, Saccharomyces cerevisiae
VAAYDFNYPIKKDSSSQLLSVQQGETIYILNKNSSGWWDGLVIDDSNGKVNRGWFPQNFGRPL

SEQ ID NO: 13 - DNA, 24 bp, Homo sapiens
atggatgcagaattccgacatgac

SEQ ID NO: 14 - DNA, 24 bp, Homo sapiens
ctagttctgcatctgctcaaagaa

(19) United States
(12) Patent Application Publication
(10) Pub. No.: US 2003/0077740 A1
(43) Pub. Date: Apr. 24, 2003

(54) Presenilin/Crk binding polypeptides (PCBP) and methods of use thereof

(76) Inventors: David R. Schubert, La Jolla, CA (US); Atsushi Kashiwa, Yokohama (JP); Hideo Kimura, Tokyo (JP)

Correspondence Address: SCHWEGMAN, LUNDBERG, WOESSNER & KLUTH, P.A., P.O. BOX 2938, MINNEAPOLIS, MN, 55402, US

(21) Appl. No.: 10/234,961

(22) Filed: Sep. 3, 2002

Related U.S. Application Data
(63) Continuation of application No. PCT/US01/07024, filed on Mar. 5, 2001.

(51) Int. Cl. C12P 21/02; C12N 5/06; C07H 21/04; C12N 9/64

(52) U.S. Cl. 435/69.1; 435/320.1; 435/226; 435/325; 536/23.2

(57) ABSTRACT

The invention provides Presenilin/Crk binding polypeptides characterized by interacting with Presenilin polypeptides, modulating intracellular calcium mobilization, and redistributing from cytoplasm to membrane in the presence of a Presenilin polypeptide. The Presenilin/Crk binding polypeptides are further characterized by interacting with a Crk polypeptide and by containing at least one SH3 domain. Also provided are isolated nucleic acids encoding Presenilin/Crk binding polypeptides, antibodies that bind to Presenilin/Crk binding polypeptides, expression vectors containing Presenilin/Crk binding polynucleotides and host cells transformed by Presenilin/Crk binding polynucleotides. The invention provides a method for identifying a compound that modulates intracellular calcium mobilization and/or modulates Presenilin/Crk binding protein levels or activity. The invention includes a method of treating a disorder associated with Presenilin/Crk binding protein. Also provided is a method of diagnosis of a subject having or at risk of having a disorder of the nervous system such as Alzheimer's disease.

### FIELD OF THE INVENTION

The present invention relates generally to neurodegenerative disorders and more specifically to a group of Presenilin/Crk binding polypeptides (PCBPs) and methods of use for modulating intracellular calcium mobilization and for diagnosing Alzheimer's disease.

### BACKGROUND OF THE INVENTION

Alzheimer's Disease (AD) is a degenerative disorder of the human central nervous system characterized by progressive memory impairment and cognitive and intellectual decline during mid to late adult life. The disease is accompanied by a variety of neuropathologic features principal among which are the presence of extracellular amyloid or senile plaques and the neurofibriliary degeneration of neurons. The etiology of this disease is complex, although in some families it appears to be inherited as an autosomal dominant trait. However, even among these inherited forms of AD, there are at least three different genes that confer inherited susceptibility to this disease. The Σ4 (Cys112Arg) allelic polymorphism of the Apolipoprotein E (ApoE) gene has been associated with AD in a significant proportion of cases with onset late in life. Similarly, a very small proportion of familial cases with onset before age 65 years have been associated with mutations in the β-amyloid precursor protein (APP) gene. A third locus (AD3) associated with a larger proportion of cases with early onset AD has recently been mapped to chromosome 14q24.3. The majority (70-80%) of heritable, early-onset AD maps to chromosome 14 and appears to result from one of more than 20 different amino-acid substitutions within h10presenilin-1 (PS1), the product of the recently identified S182 gene. A similar, although less common, AD-risk locus on chromosome 1 encodes the highly homologous h11presenilin-2. Several amino-acid substitutions have been identified within PS2 that appear to be causative for early-onset AD. Based upon mRNA detection, the presenilins appear to be ubiquitously expressed, suggesting that they are housekeeping proteins required by many cell types.

Clues regarding the biological functions of the presenilin polypeptides in non-Alzheimer brains have been obtained from studies in Caenorhabditis elegans where presenilin homologues with at least 50% identity relative to mammalian presenilin have been identified. One presenilin homologue, sel-12, interacts with genes in the Notch-signaling pathway and therefore may be involved in modulating embryonic development and cellular differentiation. Although a mammalian Notch signaling pathway and its expression protein during development have been identified, presenilin polypeptides are not expressed in the same pattern, suggesting that in mammals presenilins may have several functions in addition to a role in Notch signaling.

Presenilin 1 is a 43-45 kDa polypeptide and Presenilin 2 is a 53-55 kDa polypeptide. In brain, full-length presenilin polypeptides are not detected, although proteolytic cleavage fragments are readily identified and are found in high molecular weight complexes that are detergent sensitive. Components of the complexes in addition to the presenilin fragments are not known, however, certain proteins may be potential candidates, as they are known to interact with various domains in the amino acid sequences of Presenilin 1 and Presenilin 2. Among the putative presenilin-interacting proteins are five armadillo-related proteins: α-, β-, and γ-catenin, and neural specific plakophilin.

The functions of the interacting proteins are not well understood. Missense mutations of Presenilin 1 appear to destabilize and cause defective intracellular trafficking of β-catenin. Although none of the known interacting proteins appears to influence presenilin processing or accumulation of proteolytic fragments, differential interactions between presenilin polypeptides and proteins capable of specifically binding to presenilins may control particular roles of the normal and mutant forms of the presenilin polypeptides during development.

Calsenilin and calmyrin are both cytoplasmic proteins involved in calcium metabolism and both colocalize with presenilin when both are overexpressed (Buxbaum et al. (1998), Stabler et al. (1999)). Calmyrin interacts preferentially with PS2 and calsenilin binds to the C-terminus of both PS1 and PS2. Assuming that calsenilin, calnyrin and PBP are coexpressed in the same CNS cells, these data suggest that a relatively large macromolecular complex centered around presenilin may be involved in calcium homeostasis. Another component of this complex may be beta-catenin (Yu et al., J. Cell Biol., 273:16470-16475 (1998)). Since calcium is a major player in most nerve signaling and cell death pathways, the disruption of this complex by a variety of mechanisms could lead to the disruption of normal cell physiology.

### SUMMARY OF THE INVENTION

The present invention is based on the discovery of proteins that bind to the Alzheimer's disease-associated presenilin polypeptides and to Crk, an adapter protein containing SH2 and SH3 domains that transduce tyrosine phosphorylation signals to the Ras family of proteins. These binding proteins, referred to herein as PCBPs, for presenilin/Crk binding polypeptides, were found to be deficient in the soluble fraction of sporadic AD brains, and to accumulate in dystrophic neurons. These observations led to invention methods for diagnosis of Alzheimer's disease (AD) and other neurodegenerative diseases and provide evidence that PCBPs may be involved in the aberrant accumulation of proteins in AD neurons.

In a first embodiment, the invention provides Presenilin/Crk Binding polypeptides, polynucleotide sequences that encode PCBP polypeptides, and antibodies that are immunoreactive with the polypeptides. The amino acid sequence of Presenilin/Crk binding polypeptide contains binding domains that permit the interaction of Presenilin/Crk binding polypeptide with other polypeptides. One specific type of interacting polypeptide is a Presenilin polypeptide that is involved in the pathogenesis of early-onset Alzheimer's disease, thereby indicating a role for the Presenilin/Crk binding polypeptides in the etiology of nervous system disorders.

In one embodiment, there is provided a Presenilin/Crk binding protein characterized by interacting with a Presenilin polypeptide, modulating intracellular calcium mobilization, and redistributing from cytoplasm to membrane in the presence of a Presenilin polypeptide. The Presenilin/Crk binding polypeptide is further characterized by interacting with a Crk polypeptide and by containing at least one SH3 domain. An exemplary PCBP is shown in SEQ ID NO:2. Also included in the invention are polypeptides having at least 60% homology to SEQ ID NO:2, more preferably, at least 70% homology with SEQ ID NO:2.

There is also provided an isolated nucleic acid encoding a Presenilin/Crk binding polypeptide having the nucleotide sequence as set forth in SEQ ID NO: 1, purified Presenilin/Crk binding polypeptide having substantially the same amino acid sequence as set forth in SEQ ID NO:2, as well as biologically active fragments thereof. Also included is a polynucleotide encoding a) a polypeptide having an amino acid sequence as set forth in SEQ ID NO:2; b) a polynucleotide of a), wherein T can be U; c) a polynucleotide complementary to a) or b); d) a polynucleotide having a nucleotide sequence as set forth in SEQ ID NO:1; e) degenerate variants of a), b), c) or d); and f) a fragment of a), b), c), d) or e) having at least 15 base pairs and that hybridizes to a polynucleotide encoding a polypeptide as set forth in SEQ ID NO:2. Polynucleotides of the invention include nucleotide sequences at least 15 bases in length which hybridize under moderate to highly stringent conditions to DNA encoding a polypeptide of SEQ ID NO:2.

In another embodiment there is provided an isolated polynucleotide encoding a polypeptide characterized by interacting with a Presenilin polypeptide, modulating intracellular calcium mobilization and redistributing from cytoplasm to membrane in the presence of a Presenilin polypeptide.

In another embodiment there is provided a purified peptide having the amino acid sequence PPXLPXKR (SEQ ID NO:4) and an isolated nucleic acid encoding a peptide having the sequence PPXLPXKR (SEQ ID NO:4). Also included is a purified peptide having the amino acid sequence EERGVKLGLGDFIFYSVLVGKA (SEQ ID NO:3).

In yet another embodiment there is provided an antibody that binds to a Presenilin/Crk binding polypeptide or immunoreactive fragments of the polypeptide. Such antibodies are useful for detecting PCBP in patient samples, for example.

In still another embodiment there is provided an expression vector containing a polynucleotide sequence of the invention. An exemplary polynucleotide is set forth in SEQ ID NO:1. The invention also provides a host cell containing the expression vector. In another embodiment, the invention provides a method for producing a PCBP polypeptide of the invention or biologically active fragments thereof. An exemplary PCBP polypeptide, having the amino acid sequence of SEQ ID NO:2, is produced by culturing a host cell containing an expression vector and recovering the polypeptide from the host cell culture.

In another embodiment, the invention provides a method for diagnosing a subject having or at risk of having a neurodegenerative disorder. The method includes detecting PCBP in a soluble and an insoluble fraction of CNS tissue from the subject, wherein a level of PCBP that is higher in the insoluble fraction as compared to the soluble fraction is indicative of a neurodegenerative disorder. The method optionally includes comparing the level of PCBP in a soluble and an insoluble fraction of CNS tissue from a control subject not having a neurodegenerative disorder with the sample from the subject suspected of having or at risk of having the disorder. Preferably, the subject is a human and the CNS tissue is brain tissue.

A method of the invention also includes diagnosing a subject having or at risk of having a neurodegenerative disorder associated with PCBP by detecting PCBP level or activity in a sample CNS tissue from the subject, wherein a difference in the level or activity of PCBP in the subject as compared to a normal subject is indicative of a neurodegenerative disorder. In one aspect, the neurodegenerative disorder is Alzheimer's disease (AD).

In yet another embodiment, the invention provides a method for identifying a compound that modulates intracellular calcium mobilization. The method includes incubating the compound with a cell expressing a Presenilin/Crk binding polypeptide or fragment thereof and a presenilin and/or Crk polypeptide under conditions sufficient to permit the compound to interact with the cell; and comparing intracellular calcium mobilization in a cell incubated with the compound with the intracellular calcium mobilization in a cell not incubated with the compound, wherein a difference in calcium mobilization is indicative of a compound that modulates intracellular calcium mobilization. The cellular response may be a decrease or an increase in calcium mobilization.

The invention includes a method of treating a disorder associated with intracellular calcium mobilization by administering to a subject in need thereof a therapeutically effective amount of a compound that modulates Presenilin/Crk binding polypeptide activity or level, thereby treating the disorder.

In yet another embodiment, the invention provides a method for identifying a compound that modulates Presenilin/Crk binding polypeptide (PCBP) activity or expression. The method includes incubating components comprising the compound and PBCP or a polynucleotide encoding PCBP and presenilin and/or Crk under conditions sufficient to permit the components to interact; and determining the effect of the compound on PCBP activity or expression.

The invention also includes a kit useful for the diagnosis of a subject having or at risk of having a neurodegenerative disorder including carrier means having at least one container containing a reagent that interacts with a Presenilin/Crk binding polypeptide thereby permitting the level of Presenilin/Crk binding polypeptide to be determined.

The invention also includes a method of treating a subject having or at risk of having a disorder associated with a decreased level or activity of a Presenilin/Crk binding polypeptide, as compared to a subject not having the disorder. The method includes introducing into a subject having or at risk of having the disorder a polynucleotide encoding the Presenilin/Crk binding polypeptide operatively linked to a regulatory sequence, thereby treating the subject.

Also included is a transgenic non-human animal whose genome comprises a disruption of a Presenilin/Crk binding polypeptide gene, wherein said disruption results in the animal exhibiting a disorder of the nervous system as compared to a wild-type animal not having the disruption and a method for making the same.

The invention also provides an excision knockout mouse whose genome contains an excision of a Presenilin/Crk binding polypeptide gene, the excision being accomplished by homologous recombination and where the excision results in the mouse exhibiting a disorder of the nervous system as compared to a wild-type mouse.

The invention also includes a substantially pure Presenilin/Crk binding polypeptide, wherein the polypeptide is characterized by (a)interacting with a presenilin polypeptide, (b) modulating intracellular calcium mobilization, and (c) redistributing from cytoplasm to membrane in the presence of a presenilin polypeptide and conservative variants thereof.

The invention also provides a substantially pure Presenilin/Crk binding polypeptide, wherein the polypeptide is characterized by (a)interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide is further characterized by (a) interacting with a Crk polypeptide, and (b) containing at least one SH3 domain.

The invention provides a substantially pure Presenilin/Crk binding polypeptide, wherein the polypeptide is characterized by (a)interacting with a presenilin polypeptide, (b) modulating intracellular calcium mobilization, and (c) redistributing from cytoplasm to membrane in the presence of a presenilin polypeptide and conservative variants, wherein the Presenilin/Crk binding polypeptide has an expression pattern in the brain.

The invention also includes a substantially pure Presenilin Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide is a mammalian polypeptide.

The invention provides a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/CRK binding polypeptide is a human polypeptide.

The invention also provides a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide has a molecular weight of about 233 kD as determined by SDS-PAGE.

The invention includes a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide has at least about 60% homology to the amino acid sequence set forth in SEQ ID NO:2.

The invention also includes a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide has at least about 70% homology to the amino acid sequence set forth in SEQ ID NO:2.

The invention provides biologically active fragments of a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof.

The invention also provides substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide has a sequence substantially the same as the amino acid sequence set forth in SEQ ID NO:2.

In the invention includes a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide has a sequence as set forth in SEQ ID NO:2.

The invention provides a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide is encoded by SEQ ID NO:1 or degenerative variants thereof.

The invention also provides a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the modulating of intracellular calcium is a decrease in intracellular calcium.

The invention includes a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the modulating of intracellular calcium is an increase in intracellular calcium.

The invention also includes a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk polypeptide comprises an amino acid sequence containing residues PPXLPXKR (SEQ ID NO:4).

The invention provides a substantially pure peptide having the amino acid sequence PPXLPXKR (SEQ ID NO:4).

The invention also provides an isolated polynucleotide encoding the amino acid sequence PPXLPXKR (SEQ ID NO:4).

The invention includes an antibody that binds to a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, or to immunoreactive fragments thereof.

The invention also includes a substantially pure peptide having the amino acid sequence EERGVKLGLGDFIFYSVLVGKA (SEQ ID NO:3).

The invention provides an isolated polynucleotide encoding a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof or a substantially pure Presenilin/Crk binding polypeptide, wherein the polypeptide is characterized by (a) interacting with a presenilin polypeptide, (b) modulating intracellular calcium mobilization, and (c) redistributing from cytoplasm to membrane in the presence of a presenilin polypeptide and conservative variants thereof, wherein the Presenilin/Crk binding polypeptide is further characterized by (a) interacting with a Crk polypeptide, and (b) containing at least one SH3 domain.

The invention also provides an isolated polynucleotide selected from the group consisting of: a) a polynucleotide encoding a polypeptide having an amino acid sequence as set forth in SEQ ID NO:2; b) a polynucleotide of a), wherein T can be U; c) a polynucleotide complementary to a) or b); d) a polynucleotide having a nucleotide sequence as set forth in SEQ ID NO: 1; e) degenerate variants of a), b), c) or d); and f) a fragment of a), b), c), d) or e) having at least 15 base pairs and that hybridizes to a polynucleotide encoding a polypeptide as set forth in SEQ ID NO:2.

The invention includes an isolated polynucleotide, wherein the nucleotide is at least 15 bases in length and will hybridize under moderate to highly stringent conditions to DNA encoding a polypeptide of SEQ ID NO:2.

In the invention also includes an expression vector, wherein the expression vector comprises an isolated polynucleotide, wherein the nucleotide is at least 15 bases in length and will hybridize under moderate to highly stringent conditions to DNA encoding a polypeptide of SEQ ID NO:2.

The invention provides a host cell comprising an expression vector, wherein the expression vector comprises an isolated polynucleotide, wherein the nucleotide is at least 15 bases in length and will hybridize under moderate to highly stringent conditions to DNA encoding a polypeptide of SEQ ID NO:2.

The invention also provides a method for producing a polypeptide comprising the steps of: (a) culturing a host cell, comprising an expression vector, wherein the expression vector comprises an isolated polynucleotide, wherein the nucleotide is at least 15 bases in length and will hybridize under moderate to highly stringent conditions to DNA encoding a polypeptide of SEQ ID NO:2, under conditions suitable for the expression of the polypeptide; and (b) recovering the polypeptide from the host cell culture.

The invention includes a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder.

The invention also includes a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder and the method further comprises comparing the level of PCBP in a soluble and an insoluble fraction of CNS tissue from a control subject not having a neurodegenerative disorder with the sample from the subject suspected of having or at risk of having the disorder.

The invention provides a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder and wherein the subject is human.

The invention also includes a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder and wherein the CNS tissue is brain tissue.

The invention provides a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder and wherein the CNS tissue is cerebrospinal fluid (CSF).

The invention also provides a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder and wherein the neurodegenerative disorder is Alzheimer's disease (AD).

The invention includes a method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder and wherein the detection is by Western Blot analysis.

The invention also includes a method for diagnosing a subject having or at risk of having a neurodegenerative disorder associated with PCBP comprising detecting PCBP level or activity in a sample CNS tissue from the subject, wherein a difference in the level or activity of PCBP in the subject as compared to a normal subject is indicative of a neurodegenerative disorder.

The invention provides a method for diagnosing a subject having or at risk of having a neurodegenerative disorder associated with PCBP comprising detecting PCBP level or activity in a sample CNS tissue from the subject, wherein a difference in the level or activity of PCBP in the subject as compared to a normal subject is indicative of a neurodegenerative disorder and wherein the neurodegenerative disorder is Alzheimer's disease (AD).

The invention also provides a method for identifying a compound that modulates intracellular calcium mobilization comprising: (a) incubating the compound with a cell expressing a Presenilin/Crk binding polypeptide or fragment thereof and a presenilin and/or Crk polypeptide under conditions sufficient to permit the compound to interact with the cell; and (b) comparing intracellular calcium mobilization in a cell incubated with the compound with the intracellular calcium mobilization in a cell not incubated with the compound, wherein a difference in calcium mobilization is indicative of a compound that modulates intracellular calcium mobilization.

The invention includes a method for identifying a compound that modulates intracellular calcium mobilization comprising: (a) incubating the compound with a cell expressing a Presenilin/Crk binding polypeptide or fragment thereof and a presenilin and/or Crk polypeptide under conditions sufficient to permit the compound to interact with the cell; and (b) comparing intracellular calcium mobilization in a cell incubated with the compound with the intracellular calcium mobilization in a cell not incubated with the compound, wherein a difference in calcium mobilization is indicative of a compound that modulates intracellular calcium mobilization and wherein the cellular response is a decrease in calcium mobilization.

The invention also includes a method for identifying a compound that modulates intracellular calcium mobilization comprising: (a) incubating the compound with a cell expressing a Presenilin/Crk binding polypeptide or fragment thereof and a presenilin and/or Crk polypeptide under conditions sufficient to permit the compound to interact with the cell; and (b) comparing intracellular calcium mobilization in a cell incubated with the compound with the intracellular calcium mobilization in a cell not incubated with the compound, wherein a difference in calcium mobilization is indicative of a compound that modulates intracellular calcium mobilization and wherein the cellular response is an increase in calcium mobilization.

The invention provides a method for identifying a compound that modulates intracellular calcium mobilization comprising: (a) incubating the compound with a cell expressing a Presenilin/Crk binding polypeptide or fragment thereof and a presenilin and/or Crk polypeptide under conditions sufficient to permit the compound to interact with the cell; and (b) comparing intracellular calcium mobilization in a cell incubated with the compound with the intracellular calcium mobilization in a cell not incubated with the compound, wherein a difference in calcium mobilization is indicative of a compound that modulates intracellular calcium mobilization, wherein the compound is selected from peptides, peptidomimetics, polypeptides, pharmaceuticals, chemical compounds, biological agents, antibodies, neurotropic agents and anti-epileptic agents.

The invention provides a method of treating a disorder associated with intracellular calcium mobilization comprising administering to a subject in need thereof a therapeutically effective amount of a compound that modulates Presenilin/Crk binding polypeptide activity or level, thereby treating the disorder.

The invention also provides a method of treating a disorder associated with intracellular calcium mobilization comprising administering to a subject in need thereof a therapeutically effective amount of a compound that modulates Presenilin/Crk binding polypeptide activity or level, thereby treating the disorder, wherein the disorder is neurodegeneration, psychiatric disorders, schizophrenia, mood disorders, disorders of extrapyramidal motor function, disorders of motor control and function, attention deficit disorders, concentration disorders, pain disorders, epilepsy, convulsive disorders, eating disorders, sleep disorders, sexual disorders, circadian disorders, drug withdrawal, drug addiction, compulsive disorders, anxiety, panic disorders, or depressive disorders.

The invention includes a method of treating a disorder associated with intracellular calcium mobilization comprising administering to a subject in need thereof a therapeutically effective amount of a compound that modulates Presenilin/Crk binding polypeptide activity or level, thereby treating the disorder, wherein the disorder is Alzheimer's disease, Amyotrophic Lateral Sclerosis or Parkinson's disease.

The invention also includes a method of treating a disorder associated with intracellular calcium mobilization comprising administering to a subject in need thereof a therapeutically effective amount of a compound that modulates Presenilin/Crk binding polypeptide activity or level, thereby treating the disorder and wherein the compound comprises an agonist or antagonist of a Presenilin/Crk binding polypeptide level or activity.

The invention provides a method for identifying a compound that modulates Presenilin/Crk binding polypeptide (PCBP) activity or expression comprising: (a) incubating components comprising the compound and PBCP or a polynucleotide encoding PCBP and presenilin and/or Crk under conditions sufficient to permit the components to interact; and (b) determining the effect of the compound on PCBP activity or expression.

The invention also includes a kit useful for the diagnosis of a subject having or at risk of having a neurodegenerative disorder comprising carrier means comprising at least one container containing a reagent that interacts with a Presenilin/Crk binding polypeptide thereby permitting the level of Presenilin/Crk binding polypeptide to be determined.

The invention provides a kit useful for the diagnosis of a subject having or at risk of having a neurodegenerative disorder comprising carrier means comprising at least one container containing a reagent that interacts with a Presenilin/Crk binding polypeptide thereby permitting the level of Presenilin/Crk binding polypeptide to be determined and wherein the reagent is an antibody or fragment thereof.

The invention also provides a kit useful for the diagnosis of a subject having or at risk of having a neurodegenerative disorder comprising carrier means comprising at least one container containing a reagent that interacts with a Presenilin/Crk binding polypeptide thereby permitting the level of Presenilin/Crk binding polypeptide to be determined and wherein the kit further comprises a second container containing a buffered solution for solubilizing CNS tissue.

The invention includes a method of treating a subject having or at risk of having a disorder associated with a decreased level or activity of a Presenilin/Crk binding polypeptide, as compared to a subject not having the disorder comprising: introducing into a subject having or at risk of having the disorder a polynucleotide encoding the Presenilin/Crk binding polypeptide operatively linked to a regulatory sequence, thereby treating the subject.

The invention also includes a method of treating a subject having or at risk of having a disorder associated with a decreased level or activity of a Presenilin/Crk binding polypeptide, as compared to a subject not having the disorder comprising: introducing into a subject having or at risk of having the disorder a polynucleotide encoding the Presenilin/Crk binding polypeptide operatively linked to a regulatory sequence, thereby treating the subject, wherein the function of the Presenilin/Crk binding polypeptide is modulation of intracellular calcium concentration.

The invention provides a method of treating a subject having or at risk of having a disorder associated with a decreased level or activity of a Presenilin/Crk binding polypeptide, as compared to a subject not having the disorder comprising: introducing into a subject having or at risk of having the disorder a polynucleotide encoding the Presenilin/Crk binding polypeptide operatively linked to a regulatory sequence, thereby treating the subject, wherein the function of the Presenilin/Crk binding protein is to decrease intracellular calcium concentration.

The invention also includes a transgenic non-human animal whose genome comprises a disruption of a Presenilin/Crk binding polypeptide gene, wherein said disruption results in the animal exhibiting a disorder of the nervous system as compared to a wild-type animal not having the disruption.

The invention provides transgenic non-human animal whose genome comprises a disruption of a Presenilin/Crk binding polypeptide gene, wherein said disruption results in the animal exhibiting a disorder of the nervous system as compared to a wild-type animal not having the disruption, wherein the animal is homozygous or heterozygous for said disruption of the endogenous Presenilin/Crk binding polypeptide gene.

The invention also provides a method for producing a transgenic mouse exhibiting a disorder of the nervous system, said method comprising: (a) introducing a transgene comprising a selectable marker sequence into a mouse embryonic stem cell; (b) introducing the mouse embryonic stem cell into a mouse embryo; (c) transplanting the embryo into a pseudopregnant mouse; (d) allowing the embryo to develop to term; and (e) identifying a transgenic mouse whose genome comprises a disruption of the endogenous Presenilin/Crk binding polypeptide gene, wherein the disruption results in the mouse exhibiting a disorder of the nervous system as compared to a wild-type mouse.

The invention includes a method for producing a transgenic mouse exhibiting a disorder of the nervous system, said method comprising: (a) introducing a transgene comprising a selectable marker sequence into a mouse embryonic stem cell; (b) introducing the mouse embryonic stem cell into a mouse embryo; (c) transplanting the embryo into a pseudopregnant mouse; (d) allowing the embryo to develop to term; and (e) identifying a transgenic mouse whose genome comprises a disruption of the endogenous Presenilin/Crk binding polypeptide gene, wherein the disruption results in the mouse exhibiting a disorder of the nervous system as compared to a wild-type mouse, and wherein the transgenic mouse is homozygous or heterozygous for the disruption of the endogenous Presenilin/Crk binding polypeptide gene.

The invention provides a method of regulating the level of β-amyloid precursor protein in vitro or in vivo, as compared to an untreated cell or subject, comprising: introducing into a subject or a cell a sense or antisense polynucleotide encoding the Presenilin/Crk binding polypeptide operatively linked to a regulatory sequence.

### BRIEF DESCRIPTION OF THE DRAWINGS

FIG. 1a shows a schematic representation of PBP. The bars indicate the cDNAs which were cloned. The shaded box represents the open reading frame, and the hatched box indicates a putative SH3 domain. FIG. 1b shows amino acid sequence of PBP compared with that of Dock 180. The boxed amino acid residues correspond to a putative SH3 domain. The underlined residues indicate the consensus sequences for Crk-binding. FIG. 1e shows a comparison of SH3 domains. Residues conserved in three or more proteins are boxed. Abbreviation: PLC, phospholipase C. (PBP sequence is SEQ ID NO:8; DOCK180 sequence is SEQ ID NO:9; PLC sequence is SEQ ID NO:10; SRC sequence is SEQ ID NO:11; CDC25 sequence is SEQ ID NO:12) FIG. 1d shows a determination of the minimal sequence of PS required for the interaction with PBP in the yeast two-hybrid system. Each PS fragment fused to the GAL4-DNA binding domain was co-transformed with a plasmid containing the PBPα-GAL4 activation domain, and a β-galactosidase assay was performed. The experiment was repeated four times with similar results. FIGS. 1e and 1f show the nucleotide and deduced amino acid sequence of a Presenilin/Crk binding polypeptide, respectively. (SEQ ID NO:1 and 2, respectively).

FIG. 2b shows that PBP (MW 233,000) interacts with full length PS1 (MW 43,000) as assayed by coimmunoprecipitation. Western blot analysis with an antibody against Crk shows that PBP co-precipitates with Crk (FIG. 2a). When PBP or PS1 alone is expressed, PBP is found predominantly in the soluble fraction and PS1 in the membrane fraction (FIG. 2c). Coexpression of PBP and PS1 causes a shift in the localization of PBP to the membrane fraction (FIG. 2c). Through northern blot analysis, a single transcript of 8.6 kb was identified only in brain (FIG. 2d). No detectable mRNA was seen in heart, liver, spleen or kidney.

FIG. 3 demonstrates that PBP alone diffusely localizes in the cytoplasm (FIGS. 3A and B), while PS1 alone has an apparent ER localization (FIG. 3C). Cells transfected with both PS1 and PBP show patches of intense PBP-PS1 colocalization (FIGS. 3D, E and F).

FIG. 4 demonstrates that PBP alone slightly decreases the Ca²⁺-activated Cl⁻ current, and that co-expression of PBP and PS1 significantly suppresses the current.

FIG. 5 demonstrates that the PBP mRNA is highly localized to the cerebral cortex and hippocampus. PBP mRNA is found within the intermediate and deep layers, while only a weak signal is detected in superficial layer I (FIGS. 5a and 5e). PBP mRNA is also abundant in the piriform cortex (FIGS. 5a and g). Within the hippocampus, PBP mRNA is found in the pyramidal neurons of the CA1, CA2 and CA3 regions, and the dentate gyrus (FIGS. 5a and c). Signal is not detected with a sense probe (FIGS. 5b, d, f, and h).

FIG. 6 demonstrates that PBP in the TBS-soluble fraction of the AD brain is very much reduced relative to PBP in the TBS-soluble fraction of normal brain, while PBP is found in the TBS-insoluble fraction of both control and AD brain. Immunoblotting with antisera against PS1, synaptophysin, and 3 neurofilament subunits shows that there is no detectable degradation in the samples of AD brain compared with the brain samples form normal individuals (FIG. 6).

FIG. 7 demonstrates that PBP is localized to the dystrophic large neurons that stain with anti-tau. Double staining hippocampal slices of AD brains with anti-PBP and anti-tau demonstrate that PBP and tau frequently localize to the same dystrophic neurons.

FIGS. 8a-d demonstrates decreased sAPPα secretion in nerve cells expressing the PBP protein.

FIG. 9 demonstrates the effect of PBP on the β-secretion pathway of APP.

FIGS. 10a-d demonstrates that APP is not accumulated intracellularly in cells expressing PBP.

FIGS. 11a-c demonstrates that PBP alters the rate of intracellular APP protein degradation.

### DETAILED DESCRIPTION OF THE INVENTION

In a first embodiment, the present invention provides a substantially pure Presenilin/Crk binding polypeptide wherein the polypeptide is characterized by interacting with a Presenilin polypeptide, modulating intracellular calcium mobilization and redistributing from cytoplasm to membrane in the presence of a Presenilin polypeptide. The present invention provides for substantially pure protein preparations including polypeptides comprising or derived from the Presenilin/Crk binding polypeptides. The Presenilin/Crk binding polypeptide sequences of the invention include the specifically disclosed sequences, variants of these sequences resulting from alternative mRNA splicing, allelic variants of these sequences, muteins of these sequences and homologous or orthologous variants of these sequences.

As used herein, the term “substantially pure” refers to Presenilin/Crk binding protein that is substantially free of other proteins, lipids, carbohydrates or other materials with which it is naturally associated. One skilled in the art can purify Presenilin/Crk binding polypeptides using standard techniques for protein purification. For example, the substantially pure polypeptide will yield a single major band on a non-reducing polyacrylamide gel. The purity of the Presenilin/Crk binding protein can also be determined by amino-terminal amino acid sequence analysis or other methods known in the art.

Presenilin/Crk binding polypeptides of the invention are characterized by interacting with a presenilin polypeptide. As used herein, “Presenilin polypeptides” refers to at least two polypeptides that are encoded by genes that, in mutated forms, are associated with Familial Alzheimer's disease (see Sisodia et al. (1999) Am. J. Hum. Genet., 65:7-12). Presenilin polypeptides are expressed in many tissues including brain, and intracellularly are found associated with membrane, primarily of the endoplasmic reticulum. Two illustrative members of the Presenilin family are known as Presenilin 1 (PS1) and Presenilin 2 (PS2). PS1 and PS2 genes encode two structurally related proteins of about 43-45 kDa and about 53-55 kDa, respectively. In native cell lines and in vivo, little or no full-length PS1 and PS2 are detected, although their proteolytic cleavage fragments can be readily observed, indicating that the presenilins undergo proteolytic processing. Presenilin polypeptides contain eight transmembrane domains and the major fragments exposed to the cytoplasm are thought to be the amino terminus (amino acids 1-81), the carboxy terminus (amino acids 429-468), and a large hydrophilic loop (amino acids 263-407).

At least one site of interaction between Presenilin/Crk binding polypeptides and Presenilin polypeptides is at the hydrophilic loop domain of Presenilin polypeptides. As used herein, the term “amino acid sequence contained in a hydrophilic loop domain” refers to a loop region found between trans-membrane helices 6 and 7 of Presenilin, and extending from about amino acid 263 up to about amino acid 407. One sub-region of the hydrophilic loop, from about amino acid 376 up to about amino acid 397 of Presenilin seems to affect the interaction between Presenilin/Crk binding polypeptides and Presenilin polypeptides. The Presenilin polypeptide region of about 22 amino acids is conserved between at least two Presenilin polypeptides, PS1 and PS2. A 22 amino acid sequence mediating the interaction between a Presenilin/Crk binding polypeptide and a Presenilin polypeptide is set forth in SEQ ID NO:3 and may be designated, using the single letter code known to those in the art, as EERGVKLGLGDFIFYSVLVGKA (SEQ ID NO:3). One of skill in the art can use a sequence, such as SEQ ID NO:3, in order to perform binding studies with portions of PCBP to identify regions of PCBP that interact with the presenilin proteins. Such binding assays are well known in the art and are also described in the Examples.

As used herein, the term “interacting” or “interacts” means that two or more proteins bind to each other relatively specifically. A protein-protein interaction can be detected using a variety of molecular, biochemical and cellular techniques. For example, interactions between Presenilin/Crk binding polypeptides and Presenilin polypeptides can be detected by the yeast two-hybrid system (see Examples section). Immunoprecipitation techniques can also be used to identify polypeptides that interact with invention polypeptides. Binding may be detected by indirect functional measures reflecting the functional consequences of the interaction or by direct measures such as immunoprecipitation or alteration of protein gel electrophoresis. Other methods are described for example on pages 7-8 of PCT/CA99/00018, herein incorporated by reference. (See also Mathis, Clin. Chem., 41/9:1391, 1995; Young, K., Biol. of Reproduction, 58:302, 1998; Aujame et al., Human Antibodies, 8(4):155, 1997; Lowman, H., Ann. Rev. Biophys. Biomol. Struc., 26:401, 1997; Phizicky et al., Microbiol. Rev., 59(1):94, 1995, all of which are herein incorporated by reference).

Presenilin/Crk binding polypeptides of the invention are characterized by modulating intracellular calcium mobilization, either directly or indirectly. The term “calcium mobilization” means a change in the amount-or concentration of free calcium (Ca⁺²) sequestered in the endoplasmic reticulum, sarcoplasmic reticulum, mitochondria, or other organelles of a cell. Modulating intracellular calcium mobilization can refer to an increase or a decrease in the amount or concentration of free calcium sequestered in the endoplasmic reticulum, sarcoplasmic reticulum or mitochondria of a cell.

Presenilin/Crk binding polypeptides of the invention are also characterized by redistributing from cytoplasm to membrane in the presence of a Presenilin polypeptide. The term “redistributing” refers to a detectable change in the intracellular localization of polypeptides. Immunocytochemical studies reveal that cells transfected with plasmids containing cDNA for Presenilin/Crk binding polypeptide express Presenilin/Crk binding polypeptide in cytoplasm and cells transfected with plasmids containing Presenilin polypeptide contain Presenilin polypeptide localized to the endoplasmic reticulum. When cells are transfected with both Presenilin/Crk binding polypeptide and Presenilin polypeptide there is a Presenilin polypeptide-dependent redistribution of Presenilin/Crk binding polypeptide from the cellular cytoplasm to the membrane.

Alternatively, or in addition, Presenilin/Crk binding polypeptides of the invention can be characterized by interacting with a Crk polypeptide (see Hasegawa et al., Mol. Cell Biol., 16:1770-1776 (1996)). As used herein, the term “Crk polypeptide” refers to a polypeptide of the Crk family. A Crk polypeptide was identified originally as an oncogene product of the CT10 retrovirus and became the first example of an adaptor protein with a role in signal transduction pathways. Crk polypeptides transduce tyrosine phosphorylation signals by interacting with phosphotyrosine-containing peptides at SH2 domains. The signals are transmitted to other proteins, for example to the Ras family of proteins, which can bind to Crk at other domains Crk may be involved in signaling from focal adhesions, which not only anchor cells to the extracellular matrix, but also play a pivotal role in cell differentiation, migration and proliferation. Another function of Crk is to recruit cytoplasmic enzymes bound to a SH domain to the cell membrane. Thus, in one aspect, the invention provides methods for identifying compounds that affect Crk-associated cell differentiation, migration and proliferation by incubating test compounds with PCBP and determining an effect on Crk-associated cell differentiation, migration and proliferation (see for example Feller et al., J. Cell. Physiol., 177:535, 1998; Platanias et al., Exp. Hematol., 27:1315, 1999; Hempstead et al., Mol. and Cell. Biol., 14(3):1964, 1994). Examples of analogous screening assays are described below for presenilin-associated PCBP disorders.

Preferably, Presenilin/Crk binding polypeptides of the invention are also characterized by containing at least one SH3 domain. As used herein, “SH3 domain” refers to an amino acid sequence that mediates protein-protein interactions (Sadowski et al., Mol. Cell. Physiol., 6:4396-4408 (1986)). SH3 domains are amino acid sequences that are homologous to domains in the src family of non-receptor (cytoplasmic) protein tyrosine kinases and therefore are known as src homology domains. SH3 domains play an intermediary role in eukaryotic cellular signal transduction by virtue of their ability to bind to proteins and thus mediate transduction steps. SH3 domains bind to unmodified peptide sequences that are rich in proline and hydrophobic amino acids (Cicchetti et al., Science, 257:803-806 (1992); Ren et al., Science 259:1157-1161(1993)). SH3 domains have been identified in molecules with distinct functions in the cascade of steps following the binding of a ligand to a receptor for, among others, epidermal growth factor (EGF), platelet-derived growth factor (PDGF), insulin and interferon, and the T-cell receptor (Koch et al., Science, 252:668-674 (1991)). The key aspect of the function of SH3 domains is their ability to recognize particular amino acid sequences in their target proteins. The significant modular nature of these domains is made clear by the fact that they occur in different positions in the polypeptide chains of the intact proteins of which they are a part, and that the binding functions can often be reproduced by isolated domains.

Presenilin/Crk binding polypeptides contain an amino acid sequence which includes the residues Pro-Pro-X-Leu-Pro-X-Lys-Arg (PPXLPXKR is SEQ ID NO:4). Amino acid residues are represented by the single letter code known to those of skill in the art and amino acid residues that are variable are indicated by the letter “X”. As used herein, the term “PPXLPXKR” (SEQ ID NO:4) refers to the amino acid sequence which mediates binding of Presenilin/Crk binding polypeptide with a SH3 binding domain contained within a Crk polypeptide. At least one Presenilin/Crk binding polypeptide has two iterations of the sequence PPXLPXKR (SEQ ID NO:4) and the sequences are found in the carboxy-terminus region.

In another embodiment of the invention, there is provided a peptide having the amino acid sequence set forth in SEQ ID NO:3. Peptides containing the amino acid sequence EERGVKLGLGDFIFYSVLVGKA (SEQ ID NO:3) can be Presenilin-derived peptides. It should be understood that other proteins having all or a binding portion of SEQ ID NO:3 should be able to bind to PCBP. When it is desirable to block the interaction between PCBP and a protein containing SEQ ID NO:3, e.g., presenilin, peptides containing the amino acid sequence set forth in SEQ ID NO:3 can be used as antagonists. The term “antagonist,” as it is used herein, refers to a molecule which, when bound to PCBP, decreases the amount or the duration of the effect of the biological or immunological activity of PCBP. Antagonists may include proteins or peptides, nucleic acids, carbohydrates, antibodies, or any other molecules which decrease the effect of PCBP.

A functional Presenilin/Crk binding polypeptide includes a polypeptide as set forth in SEQ ID NO:2 and variations thereof, including conservative variations, as an illustrative polypeptide, as well as mutant or disease-causing variants of the PCBPs. The terms “conservative variation” and “substantially similar” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The terms “conservative variation” and “substantially similar” also include the use of a substituted amino acid in place of an unsubstituted parent amino acid provided that antibodies raised to the substituted polypeptide also immunoreact with the unsubstituted polypeptide. Modifications include stabilization of PCBP or of the biological activity thereof.

As used herein, the terms “protein” or “polypeptide” are used in the broadest sense to mean a sequence of amino acids that can be encoded by a cellular gene or by a recombinant nucleic acid. sequence or can be chemically synthesized. Because the proteins of the invention may be used in a variety of diagnostic, therapeutic and recombinant applications, various subsets of the PCBP sequences and combinations of the PCBP sequences with heterologous sequences are also provided. In some cases, the term “peptide” is used in referring to a portion of an amino acid sequence encoding a full length protein. A polypeptide can be a complete, fill length gene product, which can be a core protein having no amino acid modifications or can be a post-translationally modified form of a protein such as a phosphoprotein, glycoprotein, proteoglycan, lipoprotein and nucleoprotein. The terms “amino acid” or “amino acid sequence,” as used herein, refer to an oligopeptide, peptide, polypeptide, or protein sequence, or a h83fragment of any of these, and to naturally occurring or synthetic molecules. In this context, “fragments”, “immunogenic fragments”, or “antigenic fragments” refer to fragments of PCBP which are preferably about 5 to about 15 amino acids or about 5 to 50 amino acids in length, but which can be longer, and which retain some biological activity or immunological activity of PCBP. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule. For example, for use as immunogens or in binding assays, subsets of the PCBP sequences, including both normal and mutant sequences, are provided. Such protein sequences may comprise a small number of consecutive amino acid residues from the sequences which are disclosed or otherwise enabled herein but preferably include at least 4-8, and preferably at least 9-15 consecutive amino acid residues from a PCBP sequence. Other preferred subsets of the PCBP sequences include those corresponding to one or more of the functional domains or antigenic determinants of the PCBP and, in particular, may include either normal (wild-type) or mutant sequences. The invention also provides for various protein constructs in which PCBP sequences, either complete or subsets, are joined to exogenous sequences to form fusion proteins and the like. In accordance with these embodiments, the present invention also provides for methods of producing all of the above-described proteins which comprise, or are derived from, the PCBPs.

As used herein, the term “biologically active,” refers to a protein having structural, regulatory, or biochemical functions of a naturally occurring molecule. Likewise, “immunologically active” refers to the capability of the natural, recombinant, or synthetic PCBP, or of any oligopeptide thereof, to induce a specific immune response in appropriate animals or cells and to bind with specific antibodies. The term also refers to sequences including SEQ ID NO:4 and fragments of PSBP that bind to Presenilin, for example, or other Presenilin family members. The invention provides polynucleotides encoding the Presenilin/Crk binding polypeptide. These polynucleotides include DNA, cDNA and RNA sequences which encode Presenilin/Crk binding polypeptides, as well as splice variants of these sequences, allelic variants of these sequences, synonymous sequences, and homologous or orthologous variants of these sequences. It is understood that all polynucleotides encoding all or a portion of Presenilin/Crk binding polypeptides are also included herein, as long as they encode a polypeptide with Presenilin/Crk binding protein activity. Such polynucleotides include naturally occurring, synthetic, and intentionally manipulated polynucleotides. For example, Presenilin/Crk binding protein polynucleotide may be subjected to site-directed mutagenesis. The polynucleotide sequence for Presenilin/Crk binding polypeptide also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of Presenilin/Crk binding polypeptide encoded by the nucleotide sequence is functionally unchanged.

The invention includes a functional polypeptide, PCBP, and functional fragments thereof. As used herein, the term “functional polypeptide” refers to a polypeptide which possesses a biological function or activity which is identified through a defined functional assay and which is associated with a particular biologic, morphologic, or phenotypic alteration in the cell. Functional fragments of the PCBP polypeptide, include fragments of PCBP which retain the activity of PCBP. Smaller peptides containing the biological activity of PCBP are included in the invention. The biological function, for example, can vary from a polypeptide or polynucleotide fragment as small as an epitope to which an antibody molecule can bind to a large polypeptide which is capable of participating in the characteristic induction or programming of phenotypic changes within a cell. A “functional polynucleotide” denotes a polynucleotide which encodes a functional polypeptide as described herein.

The present invention also specifically provides for mutant or disease-causing variants of the PCBPs. Because the nucleic acids of the invention may be used in a variety of diagnostic, therapeutic and recombinant applications, various subsets of the PCBP sequences and combinations of the PCBP sequences with heterologous sequences are also provided. For example, for use in allele-specific hybridization screening or PCR amplification techniques, subsets of the PCBP sequences, including both sense and antisense sequences, and both normal and mutant sequences, as well as intronic, exonic and untranslated sequences, are provided. Such sequences may comprise a small number of consecutive nucleotides from the sequences which are disclosed or otherwise enabled herein but preferably include at least 8-10, and more preferably 9-25, consecutive nucleotides from a PCBP sequence. Other preferred subsets of the PCBP sequences include those encoding one or more of the functional domains or antigenic determinants of the PCBP and, in particular, may include either normal (wild-type) or mutant sequences. The invention also provides for various nucleic acid constructs in which PCBP sequences, either complete or subsets, are operably joined to exogenous sequences to form cloning vectors, expression vectors, fusion vectors, transgenic constructs, and the like.

An exemplary polynucleotide encoding a Presenilin/Crk binding polypeptide is set forth as SEQ ID NO: 1, or fragments thereof. The term “polynucleotide”, “nucleic acid”, “nucleic acid sequence”, or “nucleic acid molecule” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant nucleic acid construct which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In this context, “fragments” refers to those nucleic acid sequences which are greater than about 15 to 60 nucleotides in length, and more preferably are at least about 100 nucleotides, at least about 1000 nucleotides, or at least about 10,000 nucleotides in length. Examples of nucleic acid fragments include nucleic acid sequences encoding SEQ ID NO:4, for example, or encoding PSBP fragments that bind to Presenilin, Crk or related polypeptides, for inclusion in fusion proteins, as inhibitors or mimetics of PCBP or immunogens.

The term “homology,” as used herein, refers to a degree of complementarity. There may be partial homology or complete homology. A partially complementary sequence that at least partially inhibits an identical sequence from hybridizing to a target nucleic acid is referred to as “substantially homologous.” The inhibition of hybridization of the completely complementary sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The terms “complementary” or “complementarity,” as used herein, refer to the natural binding of polynucleotides under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single-stranded molecules. The degree of complementarity between nucleic acid strands has significant effects on the efficiency and strength of the hybridization between the nucleic acid strands. This is of particular importance in amplification reactions, which depend upon binding between nucleic acid strands, and in the design and use of peptide nucleic acid (PNA) molecules.

The phrases “percent identity” or “% identity” refer to the percentage of sequence similarity found in a comparison of two or more amino acid or nucleic acid sequences. Percent identity can be determined electronically, e.g., by using the MEGALIGN program (DNASTAR, Inc., Madison, Wis.). The MEGALIGN program can create alignments between two or more sequences according to different methods, e.g., the clustal method. (See, e.g., Higgins, D. G. and P. M. Sharp (1988) Gene, 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, tines one hundred. Gaps of low or of no homology between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol., 183:626-645.) Identity between sequences can also be determined by other methods known in the art, e.g., by varying hybridization conditions.

Presenilin/Crk binding polypeptides have an expression pattern in the brain. As used herein, the term “expression pattern” refers to tissue and cellular sites where polypeptides or polynucleotides are localized. The cellular localization of polypeptides can be determined by various methods including, for example, immunocytochemical, in situ hybridization and biochemical methods (see Examples section). For example, studies using Northern blot analysis reveal that Presenilin/Crk binding polypeptides are found in brain tissue, including the hippocampus, the cerebral cortex and the piriform cortex. In the cerebral cortex, Presenilin/Crk binding polypeptide is found within the intermediate and deep layers, with low levels in the superficial layer. Within the hippocampus, Presenilin/Crk binding polypeptide is found in the pyramidal neurons and the dentate gyrus.

Presenilin/Crk binding polypeptides are expressed in dystrophic neurons of a subject diagnosed with Alzheimer's disease. Alzheimer's disease is a neurodegenerative disease characterized by atrophy of nerve cells in the cerebral cortex, subcortical areas, and hippocampus and the presence of plaques, dystrophic neurites and neurofibrillary tangles. In Alzheimer's disease, dystrophic or aberrant neurite growth, synapse loss, and neurofibrillary tangle formation are strong correlates of disease severity. Dystrophic neurons characteristically contain abundant electrodense multilaminar bodies in the cytoplasm of the neurites and have disruption of synaptic junctions. Those of skill in the art can readily identify dystrophic neurons using ultrastructural analysis techniques.

Although it is generally understood that Alzheimer's Disease is a neurological disorder, most likely in the brain, expression of Presenilin proteins has been found in a variety of human tissues such as heart, brain, placenta, lung, liver, skeletal muscle, kidney and pancreas. Although Presenilin-family genes are expressed widely, the clinically apparent phenotype exists in brain although it is conceivable that biochemical phenotypes may exist in these other tissues. As with other genetic diseases such as Huntington's Disease and APP—Alzheimer's, the clinical disease manifestation may reflect the different biochemistries of different cell types and tissues (which stem from genetics and the protein). Such findings suggest that AD may not be solely a neurological disorder but may also be a systemic disorder, hence requiring alternative therapeutic strategies which may be targeted to other tissues or organs or generally in addition or separately from neuronal or brain tissues.

The exemplary Presenilin/Crk binding polypeptide of the invention, as set forth in SEQ ID NO:2 is a protein of 2027 amino acids. As used herein, the term “amino acid” is used in its broadest sense to mean the naturally occurring amino acids as well as non-naturally occurring amino acids, including amino acid analogs. Thus, reference herein to an amino acid includes, for example, naturally occurring (L)-amino acids, as well as (D)-amino acids, chemically modified amino acids such as amino acid analogs, and chemically synthesized compounds having properties known in the art to be characteristic of an amino acid. The number of amino acids in a polypeptide can be determined by analytical methods known to those of skill in the art.

The exemplary Presenilin/Crk binding polypeptide of the invention has a molecular mass of about 233 kD. The apparent molecular mass of a Presenilin/Crk binding protein can be measured using routine methods such as polyacrylamide gel electrophoresis performed in the presence of sodium dodecyl sulfate (SDS-PAGE) or column chromatography performed under reducing and denaturing conditions. It is recognized that the apparent molecular mass of a previously unknown protein as determined, for example, by SDS-PAGE is an estimate based on the relative migration of the unknown protein as compared to the migration of several other proteins having known molecular masses. Thus, one investigator reasonably can estimate, for example, that an unknown protein has an apparent molecular mass of 53 kD, whereas a second investigator, looking at the same unknown protein under substantially similar conditions, reasonably can estimate that the protein has an apparent molecular mass of 55 kD.

The invention also provides antibodies that bind to PCBPs or fragments thereof of the invention. Such antibodies may prevent interactions of the Presenilin/Crk binding polypeptides with presenilin, Crk, or other proteins. The term “antibody” as used in this invention includes intact molecules as well as fragments thereof, such as Fab, F(ab′)2, and Fv which are capable of binding to an epitopic determinant present in an invention polypeptide. Such antibody fragments retain some ability to selectively bind with its antigen or receptor. Binding of antibodies to Presenilin/Crk binding polypeptides can interfere with cell-signaling by interfering with an intracellular signaling pathway. Binding of antibodies can interfere with Presenilin/Crk binding polypeptide binding to Presenilin polypeptides, and the like. Binding of antibodies to Presenilin/Crk binding polypeptides can interfere with Presenilin/Crk binding polypeptides binding with Crk polypeptides. The invention includes antibodies immunoreactive with PCBP polypeptide (SEQ ID NO:3) or immunoreactive fragments thereof. Antibody which consists essentially of pooled monoclonal antibodies with different epitopic specificities, as well as distinct monoclonal antibody preparations are provided.

(1) An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

(2) An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

(3) An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds.

(4) An Fv fragment is defined as a genetically engineered fragment containing the variable region of a light chain and the variable region of a heavy chain expressed as two chains.

(5) A single chain antibody (“SCA”) is a genetically engineered single chain molecule containing the variable region of a light chain and the variable region of a heavy chain, linked by a suitable, flexible polypeptide linker.

As used in this invention, the term “epitope” refers to an antigenic determinant on an antigen, such as a PCBP polypeptide, to which the paratope of an antibody, such as an PCBP-specific antibody, binds. Antigenic determinants usually consist of chemically active surface groupings of molecules, such as amino acids or sugar side chains, and can have specific three-dimensional structural characteristics, as well as specific charge characteristics.

As is mentioned above, antigens that can be used in producing PCBP-specific antibodies include PCBP polypeptides or PCBP polypeptide fragments. The polypeptide or peptide used to immunize an animal can be obtained by standard recombinant, chemical synthetic, or purification methods. As is well known in the art, in order to increase immunogenicity, an antigen can be conjugated to a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). In addition to such carriers, well known adjuvants can be administered with the antigen to facilitate induction of a strong immune response.

The antibodies of the invention can be used in any subject in which it is desirable to administer in vitro or in vivo immunodiagnosis or immunotherapy. The antibodies of the invention are suited for use, for example, in immunoassays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The term “immunometric assay” or “sandwich immunoassay”, includes simultaneous sandwich, forward sandwich and reverse sandwich immunoassays. These terms are well understood by those skilled in the art. Those of skill will also appreciate that antibodies according to the present invention will be useful in other variations and forms of assays which are presently known or which may be developed in the future. These are intended to be included within the scope of the present invention.

Monoclonal antibodies can be bound to many different carriers and used to detect the presence of PCBP. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

In performing the assays it may be desirable to include certain “blockers” in the incubation medium (usually added with the labeled soluble antibody). The “blockers” are added to assure that non-specific proteins, proteases, or anti-heterophilic immunoglobulins to anti-PCBP immunoglobulins present in the experimental sample do not cross-link or destroy the antibodies on the solid phase support, or the radiolabeled indicator antibody, to yield false positive or false negative results. The selection of “blockers” therefore may add substantially to the specificity of the assays described in the present invention.

It has been found that a number of nonrelevant (i.e., nonspecific) antibodies of the same class or subclass (isotype) as those used in the assays (e.g., IgG1, IgG2a, IgM, etc.) can be used as “blockers”. The concentration of the “blockers” (normally 1-100 μg/μl) may be important, in order to maintain the proper sensitivity yet inhibit any unwanted interference by mutually occurring cross reactive proteins in the specimen. Methods of making these fragments are known in the art. (See for example, Harlow and Lane, Antibodies: A Laboratory Manual, Cold Spring Harbor Laboratory, New York (1988), incorporated herein by reference). Monoclonal antibodies are made from antigen containing fragments of the protein by methods well known to those skilled in the art (Kohler et al., Nature, 256:495, 1975).

Antibodies that bind to polypeptides of the invention can be prepared using an intact polypeptide or fragments containing small peptides of interest as the immunizing antigen. For example, it may be desirable to produce antibodies that specifically bind to the N- or C-terminal domains of an invention polypeptide. The polypeptide or peptide used to immunize an animal is derived from translated cDNA or chemically synthesized and can be conjugated to a carrier protein, if desired. Commonly used carrier proteins which may be chemically coupled to the immunizing peptide include keyhole limpet hemocyanin (KLH), thyroglobulin, bovine serum albumin (BSA), tetanus toxoid, and the like.

Invention polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See, for example, Coligan et al., Unit 9, Current Protocols in Immunology, Wiley Interscience, 1994).

The antibodies of the invention can be bound to many different carriers and used to detect the presence of an antigen comprising the polypeptides of the invention. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction For example, it is common to use such haptens as biotin, which reacts with avidin, or dinitrophenyl, puridoxal, and fluorescein, which can react with specific antihapten antibodies.

In using the monoclonal and polyclonal antibodies of the invention for the in vivo detection of antigen, e.g., a Presenilin/Crk binding polypeptide, the detectably labeled antibody is given a dose which is diagnostically effective. The term “diagnostically effective” means that the amount of detectably labeled antibody is administered in sufficient quantity to enable detection of the site having the antigen comprising a polypeptide of the invention for which the antibodies are specific.

The concentration of detectably labeled antibody which is administered should be sufficient such that the binding to those cells having the polypeptide is detectable compared to the background. Further, it is desirable that the detectably labeled antibody be rapidly cleared from the circulatory system in order to give the best target-to-background signal ratio.

As a rule, the dosage of detectably labeled antibody for in vivo treatment or diagnosis will vary depending on such factors as age, sex, and extent of disease of the individual. Such dosages may vary, for example, depending on whether multiple injections are given, antigenic burden, and other factors known to those of skill in the art.

In using a monoclonal antibody for the in vivo detection of antigen, the detectably labeled monoclonal antibody is given in a dose which is diagnostically effective. The term “diagnostically effective” means that the amount of detectably labeled monoclonal antibody is administered in sufficient quantity to enable detection of the site having the PCBP antigen for which the monoclonal antibodies are specific. The concentration of detectably labeled monoclonal antibody which is administered should be sufficient such that the binding to those cells having PCBP is detectable compared to the background. Further, it is desirable that the detectably labeled monoclonal antibody be rapidly cleared from the circulatory system in order to give the best target-to-background signal ratio.

As a rule, the dosage of detectably labeled monoclonal antibody for in vivo diagnosis will vary depending on such factors as age, sex, and extent of disease of the individual. The dosage of monoclonal antibody can vary from about 0.001 mg/m² to about 500 mg/m², preferably 0.1 mg/M² to about 200 mg/M², most preferably about 0.1 mg/m² to about 10 mg/M². Such dosages may vary, for example, depending on whether multiple injections are given, tumor burden, and other factors known to those of skill in the art.

For in vivo diagnostic imaging, the type of detection instrument available is a major factor in selecting a given radioisotope. The radioisotope chosen must have a type of decay which is detectable for a-given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 keV range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are 111 In, 97 Ru, 67 Ga, 68 Ga, 72 As, 89 Zr, and 201T1.

A monoclonal antibody useful in the method of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes ale used for camera imaging and paramagnetic isotopes for MRI. Elements which are particularly useful in such techniques include 157Gd, 55 Mn, 162 Dy, 52Cr, and 56 Fe.

In the present invention, the Presenilin/Crk binding protein polynucleotide sequences may be inserted into a recombinant expression vector. The term “expression vector” refers to a plasmid, virus or other vehicle known in the art that has been manipulated by insertion or incorporation of the Presenilin/Crk binding protein polynucleotide sequences. Such expression vectors contain a promoter sequence which facilitates the efficient transcription of the inserted genetic sequence of the host. The expression vector typically contains an origin of replication, a promote, as well as specific genes which allow phenotypic selection of the transformed cells. Vectors suitable for use in the present invention include, but are not limited to the T7-based expression vector for expression in bacteria (Rosenberg et al., Gene, 56:125 (198780), the pMSXND expression vector for expression in mammalian cells (Lee and Nathans, J. Bio. Chem., 263:3521 (1988)) and baculovirus-derived vectors for expression in insect cells. The DNA segment can be present in the vector operably linked to regulatory elements, for example, a promoter (e.g., T7, metallothionein I, or polyhedrin promoters). Such expression vectors can be utilized, for example, to produce a protein of the invention in vitro. The expression vector is introduced into a suitable host cell and cultured under conditions that allow expression of the polynucleotide. Expression vectors are also useful, for example, for in vivo uses such as gene therapy.

In general, an expression vector contains the expression elements necessary to achieve, for example, sustained transcription of the nucleic acid molecule, although such elements also can be inherent to the nucleic acid molecule cloned into the vector. In particular, an expression vector contains or encodes a promoter sequence, which can provide constitutive or, if desired, inducible expression of a cloned nucleic acid sequence, a poly-A recognition sequence, and a ribosome recognition site, and can contain other regulatory elements such as an enhancer, which can be tissue specific. The vector also contains elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol. Vol 185, D. V. Goeddel, ed. (Academic Press, Inc., 1990); Jolly, Canc. Gene Ther., 1:51-64 (1994); Flotte, J. Bioenerg. Biomemb. 25:3742 (1993); Kirshenbaum et al., J. Clin. Invest. 92:381-387 (1993), which is incorporated herein by reference).

In particular, an expression vector contains a promoter sequence, which can provide constitutive or, if desired, inducible expression of the encoding nucleic acid molecule, and a poly-A recognition sequence, and can contain other regulatory elements such as an enhancer, which can be tissue specific.

Similarly, a eukaryotic expression vector can include, for example, a heterologous or homologous RNA transcription promoter for RNA polymerase binding, a polyadenylation signal located downstream of the coding sequence, an AUG start codon in the appropriate frame and a termination codon to direct detachment of a ribosome following translation of the transcribed mRNA.

In general, expression vectors containing promoter sequences which facilitate the efficient transcription of the inserted genetic sequence are used in connection with the host. As described above, biologically functional viral or plasmid DNA vectors capable of expression and replication in a host are known in the art. Such vectors are used to incorporate encoding DNA sequences of the invention. Expression vectors typically contain an origin of replication, a promoter, and a terminator, as well as specific genes that are capable of providing phenotypic selection of the transformed cells.

Polynucleotide sequences encoding Presenilin/Crk binding polypeptides can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Methods that are well known to those skilled in the art can be used to construct expression vectors containing the Presenilin/Crk binding polypeptide coding sequence and appropriate transcriptional/translational control signals. These methods include in vitro recombinant DNA techniques, synthetic techniques, and in vivo recombinant/genetic techniques. See, for example, the techniques described in Maniatis et al., 1989 Molecular cloning, A Laboratory Manual, Cold Spring Harbor Laboratory, N.Y.

A method is provided for producing a polypeptide containing the amino acid sequence of SEQ ID NO:2 or fragments thereof, including culturing the host cell under conditions suitable for the expression of the polypeptide and recovering the polypeptide from the host cell culture.

A Presenilin/Crk binding polypeptide or a fragment thereof, can be encoded by a recombinant or non-recombinant nucleic acid molecule and expressed in a cell. Preparation of a Presenilin/Crk binding polypeptide by recombinant methods provides several advantages. In particular, the nucleic acid sequence encoding the Presenilin/Crk binding polypeptide can include additional nucleotide sequences encoding, for example, peptides useful for recovering the Presenilin/Crk binding polypeptide from the host cell. A Presenilin/Crk binding polypeptide can be recovered using well-known methods, including, for example, precipitation, gel filtration, ion exchange, reverse-phase, or affinity chromatography (see, for example, Deutscher et al., Guide to Protein Purification” in Meth. Enzymol. Vol. 182 (Academic Press, 1990)). Such methods also can be used to purify a fragment of a Presenilin/Crk binding polypeptide, for example, a particular binding sequence, from a cell in which it is naturally expressed.

A recombinant nucleic acid molecule encoding a Presenilin/Crk binding polypeptide or a fragment thereof can include, for example, a protease site, which can facilitate cleavage of the Presenilin/Crk binding polypeptide from a non-Presenilin/Crk binding polypeptide sequence, for example, a tag peptide, secretory peptide, or the like. As such, the recombinant nucleic acid molecule also can encode a tag peptide such as a polyhistidine sequence, a FLAG peptide (Hopp et al., Biotechnology, 6:1204 (1988)), a glutathione S-transferase polypeptide or the like, which can be bound by divalent metal ions, a specific antibody (U.S. Pat. No. 5,011,912), or glutathione, respectively, thus facilitating recovery and purification of the Presenilin/Crk binding polypeptide comprising the peptide tag. Such tag peptides also can facilitate identification of the Presenilin/Crk binding polypeptide through stages of synthesis, chemical or enzymatic modification, linkage, or the like. Methods for purifying polypeptides comprising such tags are well known in the art and the reagents for performing such methods are commercially available.

A nucleic acid molecule encoding a Presenilin/Crk binding polypeptide can be engineered to contain one or more restriction endonuclease recognition and cleavage sites, which can facilitate, for example, substitution of an element of the Presenilin/Crk binding polypeptide such as the selective recognition domain, or where present, a spacer element. As such, related Presenilin/Crk binding polypeptides can be prepared, each having a similar activity, but having specificity for different function-forming contexts. A restriction endonuclease site also can be engineered into (or out of) the sequence coding a peptide portion of the Presenilin/Crk binding polypeptide, and can, but need not change one or more amino acids encoded by the particular sequence. Such a site can provide a simple means to identify the nucleic acid sequence, based on cleavage (or lack of cleavage) following contact with the relevant restriction endonuclease, and, where introduction of the site changes an amino acid, can further provide advantages based on the substitution.

In another embodiment of the invention, a method is provided for diagnosis of a subject having or at risk of having a disorder of the nervous system. A preferred embodiment of the present invention, includes a method for detecting in the subject a level or activity of a Presenilin/Crk binding polypeptide wherein a difference in the level or activity as compared to a normal subject is indicative of a disorder of the nervous system. In one aspect, the invention provides a method for diagnosing a subject having or at risk of having a neurodegenerative disorder including detecting PCBP in a soluble and an insoluble fraction of central nervous system (CNS) tissue from the subject, wherein a level of PCBP that is higher in the insoluble fraction as compared to the soluble fraction is indicative of a neurodegenerative disorder. CNS tissue may include brain tissue, cerebrospinal fluid or other tissue from the CNS.

Techniques for collecting cerebrospinal fluid (CSF) are well known in the art (see, e.g., Appleyard et al., 1987, Brain, 110:1309; and Wester et al., 1990, J. Neurochem., 54:1148). In the present example, CSF was collected from the lateral ventricular of the brain using a 16-gauge spinal needle within 24 hours after death. Cellular debris in the sample was removed by centrifuge at 15,000×g. Lumbar CSF can be isolated by the use of a similar technique (see, e.g., The Merck Manual, 1746-1748, 12th edition, D. N. Holvey, ed., Merck, Sharp, and Dohne Research Publishing, New Jersey, 1972). Analysis of lumbar CSF is particularly useful, as the sample can be obtained from a living patient. The CSF sample can be examined by aforementioned immunoassays for the amount and isoformal profile of PCBP, for example. PCBP in CSF can be quantified by Western blot analysis, for example, using the monoclonal antibodies of the invention.

In one aspect of the method, the amount of PCBP or the amount of biologically active PCBP present in the patient s CSF is determined relative to the amount or activity of PCBP present in a control sample of the same type (e.g., lumbar CSF or ventricular CSF) from an unaffected patient, e.g., a human. A substantial decrease of the PCBP level or activity in CSF from the patient relative to the control indicates a diagnosis of a neurodegenerative disease, e.g., Alzheimer's disease.

Alternatively, other CNS tissue, such as brain tissue can be analyzed for PCBP level or activity. The Examples show a method for preparing soluble and insoluble fractions of brain tissue, where the level of PCBP in the Tris-buffered saline (TBS) soluble fraction of a patient with Alzheimer's disease was much reduced from the level in the insoluble fraction from the same patient. In contrast, a patient not having the disease, has relatively equal amounts of PCBP in the soluble and insoluble fractions. Since a patient not having the disorder would have relatively equal levels of PCBP in each of the fractions, it is optional to use control fractions for a “normal” subject.

Additional diagnostic uses for oligonucleotides designed from the sequences encoding PCBP may involve the use of PCR. These oligomers may be chemically synthesized, generated enzymatically, or produced in vitro. Oligomers will preferably contain a fragment of a polynucleotide encoding PCBP, and will be employed under optimized conditions for identification of a specific gene or condition. Oligomers may also be employed under less stringent conditions for detection or quantitation of closely related DNA or RNA sequences.

Methods which may also be used to quantitate the expression of PCBP include radiolabeling or biotinylating nucleotides, coamplification of a control nucleic acid, and interpolating results from standard curves. The speed of quantitation of multiple samples may be accelerated by running the assay in an ELISA format where the oligomer of interest is presented in various dilutions and a spectrophotometric or calorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Such assays may also be used to evaluate the efficacy of a particular therapeutic treatment regimen in animal studies, in clinical trials, or to monitor the treatment of an individual patient.

The PCBP and PCBP-related genes and gene products, as well as other products derived therefrom (e.g., probes, antibodies), will be useful in the diagnosis of Alzheimer's Disease, presenile and senile dementias, psychiatric diseases such as schizophrenia, depression, and neurologic diseases such as stroke and cerebral hemorrhage for example. Diagnosis of inherited cases of these diseases can be accomplished by methods based upon the nucleic acids (including genomic and mRNA/cDNA sequences), proteins, and/or antibodies disclosed and enabled herein. Preferably, the methods and products are based upon the human PCBP or nucleic acids, proteins or antibodies disclosed herein. As will be obvious to one of ordinary skill in the art, however, the significant evolutionary conservation of large portions of the PCBP nucleotide and amino acid sequences, allow the skilled artisan to make use of such non-human PCBP-homologue nucleic acids, proteins and antibodies even for applications directed toward human or other mammalian subjects. Thus, for brevity but without limiting the scope of the invention, the descriptions herein focus upon uses of the human homologues of PCBP. It will be understood, however, that homologous sequences from other species, including those disclosed herein, will be equivalent for many purposes.

As will be appreciated by one of ordinary skill in the art, the choice of diagnostic methods of the present invention will be influenced by the nature of the available biological samples to be tested and the nature of the information required. PCBP, for example is highly expressed in brain tissue but brain biopsies are invasive and expensive procedures, particularly for routine screening. Assays based upon a subject's genomic DNA may be a preferred method for PCBP genetic diagnostics as no information will be lost due to alternative splicing and because essentially any nucleate cells may provide a usable sample. Diagnostics based upon other PCBP-related proteins are subject to similar considerations: availability of tissues, levels of expression in various tissues, and alternative translation products resulting from alternative mRNA splicing. Thus, screens and diagnostics based upon PCBPs are provided which detect differences between mutant and normal PCBPs in electrophoretic mobility, in proteolytic cleavage patterns, in molar ratios of the various amino acid residues, in ability to bind specific antibodies. In addition, screens and diagnostics based upon nucleic acids (gDNA, cDNA or mRNA) are provided which detect differences in nucleotide sequences by direct nucleotide sequencing, hybridization using allele specific oligonucleotides, restriction enzyme digest and mapping (e.g., RFLP, REF-SSCP), electrophoretic mobility (e.g., SSCP, DGGE), PCR mapping, Rnase protection, chemical mismatch cleavage, ligase-mediated detection, and various other methods. Other methods are also provided which detect abnormal processing of PCBPs, or proteins reacting with PCBPs (e.g., abnormal phosphorylation, glycosylation, glycation amidation or proteolytic cleavage) alterations in PCBP transcription, translation, and post-tanslational modification; alterations in the intracellular and extracellular trafficking of PCBP gene products; or abnormal intracellular localization of PCBPs. In accordance with these embodiments, diagnostic kits are also provided which will include the reagents necessary for the above-described diagnostic screens.

When a diagnostic assay is to be based upon PCBP-related proteins, a variety of approaches are possible. For example, diagnosis can be achieved by monitoring differences in the electrophoretic mobility of normal and mutant proteins. Such an approach will be particularly useful in identifying mutants in which charge substitutions are present, or in which insertions, deletions or substitutions have resulted in a significant change in the molecular mass of the resultant protein. Alternatively, diagnosis may be based upon differences in the proteolytic cleavage patterns of normal and mutant proteins, differences in molar ratios of the various amino acid residues, or by functional assays demonstrating altered function of the gene products. In some preferred embodiments, protein-based diagnostics will employ differences in the ability of antibodies to bind to normal and mutant PCBP-related proteins. Such diagnostic tests may employ antibodies which bind to the normal proteins but not to mutant proteins, or vice versa. In particular, an assay in which a plurality of monoclonal antibodies, each capable of binding to a mutant epitope, may be employed. The levels of anti-mutant antibody binding in a sample obtained from a test subject (visualized by, for example, radiolabeling, ELISA or chemiluminescence) may be compared to the levels of binding to a control sample Such antibody diagnostics may be used for in situ immunohistochemistry using biopsy samples of CNS tissues obtained antemortem or postmortem, including neuropathological structures associated with these diseases such as neurofibrillary tangles and amyloid plaques, or may be used with fluid samples such as cerebrospinal fluid or with peripheral tissues such as white blood cells.

When the diagnostic assay is to be based upon nucleic acids from a sample, either mRNA or genomic DNA may be used. When mRNA is used from a sample, many of the same considerations apply with respect to source tissues and the possibility of alternative splicing. That is, there may be little or no expression of transcripts unless appropriate tissue sources are chosen or available, and alternative splicing may result in the loss of some information. With either mRNA or DNA, standard methods well known in the art may be used to detect the presence of a particular sequence either in situ or in vitro (see, e.g., Sambrook et al., eds. (1989) Molecular Cloning: A Laboratory Manual, Cold Spring Harbor Press, Cold Spring Harbor, N.Y.).

For in situ detection of a mutant PCBP, or other PCBP-related nucleic acid sequence, a sample of tissue may be prepared by standard techniques and then contacted with a probe, preferably one which is labeled to facilitate detection, and an assay for nucleic acid hybridization is conducted under stringent conditions which permit hybridization only between the probe and highly or perfectly complementary sequences. When a PCBP mutation consists of a single nucleotide substitution, high stringency hybridization conditions will be required to distinguish normal sequences from most mutant sequences. As an example only, the following procedure may be employed on a subject: A rat animal model is anesthetized and transcardially perfused with cold PBS, followed by perfusion with a formaldehyde solution. The brain or other tissue of interest is then removed, frozen in liquid nitrogen, and cut into thin micron sections. The sections are placed on slides and incubated in proteinase K. Following rinsing in DEP, water and ethanol, the slides are placed in prehybridization buffer. A radioactive probe corresponding to the selected oligonucleotide is incubated with the sectioned brain tissue. After incubation and air drying, the labeled areas are visualized by autoradiography. Dark spots on the tissue sample indicate hybridization of the probe with brain mRNA, demonstrating expression of the nucleic acid sequence.

A significant advantage of the use of either DNA or mRNA is the ability to amplify the amount of genetic material using the polymerase chain reaction (PCR), either alone (with genomic DNA) or in combination with reverse transcription (with mRNA to produce cDNA).

In another series of embodiments, the present invention provides methods of screening or identifying proteins, small molecules or other compounds which are capable of inducing or inhibiting the expression of PCBPs. The assays may be performed in vitro using transformed or non-transformed cells, immortalized cell lines, or in vivo using the transgenic animal models or human subjects enabled herein. In particular, the assays may detect the presence of increased or decreased expression of PCBP or other PCBP-related genes or proteins on the basis of increased or deceased mRNA expression, increased or decreased levels of PCBP-related protein products, or increased or decreased levels of expression of a marker gene (e.g., β-galactosidase, green fluorescent protein, alkaline phosphatase or luciferase) operably joined to a PCBP 5′ regulatory region in a recombinant construct.

In another series of embodiments, the present invention provides methods for identifying proteins and other compounds which bind to, or otherwise directly interact with, the PCBPs. The proteins and compounds will include endogenous cellular components which interact with the PCBPs in vivo and which, therefore, provide new targets for pharmaceutical and therapeutic interventions, as well as recombinant, synthetic and otherwise exogenous compounds which may have PCBP binding capacity and, therefore, may be candidates for pharmaceutical agents. Thus, in one series of embodiments, cell lysates or tissue homogenates (e.g., human brain homogenates, lymphocyte lysates) may be screened for proteins or other compounds which bind to one of the normal or mutant PCBPs. Alternatively, any of a variety of exogenous compounds, both naturally occurring and/or synthetic (e.g., libraries of small molecules or peptides), may be screened for PCBP binding capacity. Binding may be detected by non-specific measures (e.g., changes in intracellular Ca²⁺, NA⁺, K⁺, or GTP/GCP ratio, changes in apoptosis or microtubule-associated protein phosphorylation, changes in the expression or activity of Presenilin or Crk, changes in the expression of other downstream genes which can be monitored by differential display, 2D gel electrophoresis, differential hybridization, or SAGE methods) or by direct measures such as immunoprecipitation, the Biomolecular Interaction Assay (BIAcore) or alteration of protein gel electrophoresis. The preferred methods involve variations on the following techniques: direct extraction by affinity chromatography; co-isolation of PCBP components and bound proteins or other compounds by immunoprecipitation; BIAcore analysis; the yeast two-hybrid systems, and the like.

In another series of embodiments, the present invention provides for methods of identifying proteins, small molecules and other compounds capable of modulating the activity of normal or mutant PCBPs. Using normal cells or animals, the transformed cells and animal models of the present invention, or cells obtained from subjects bearing normal or mutant PCBP genes, the present invention provides methods of identifying such compounds on the basis of their ability to affect the expression of the PCBPs, the intracellular localization of the PCBPs, changes in intracellular Ca²⁺, Na⁺, K⁺, or GTP/GDP ratios, or other ion levels or metabolic measures, the occurrence or rate of apoptosis or cell death, the levels/pattern/activities of Presenilin or Crk, the presence or levels of phosphorylation of microtubule-associated proteins, or other biochemical, histological, or physiological markers which distinguish cells bearing normal and mutant PCBP sequences. Using the animal models of the invention, methods of identifying such compounds are also provided on the basis of the ability of the compounds to affect behavioral, physiological or histological phenotypes associated with mutations in the PCBPs.

A method is provided for identifying a compound that modulates intracellular calcium mobilization. The method includes incubating the compound and a cell expressing a Presenilin/Crk binding polypeptide or fragments thereof and a Presenilin polypeptide or Crk polypeptide, under conditions sufficient to permit the compound to interact with the cell, exposing the cell to conditions sufficient to activate calcium mobilization, and comparing the cellular response in a cell incubated with the compound with the cellular response of a cell not incubated with the compound.

The cell may be any cell of interest, including but not limited to neuronal cells, glial cells, cardiac cells, bronchial cells, uterine cells, testicular cells, liver cells, renal cells, intestinal cells, cells from the thymus and spleen, placental cells, endothelial cells, endocrine cells including thyroid, parathyroid, pituitary and the like, smooth muscle cells and skeletal muscle cells. The cell is exposed to conditions sufficient to activate calcium mobilization. The effect of the compound on the cellular response is determined, either directly or indirectly, and a cellular response is then compared with a cellular response of a control cell. A suitable control includes, but is not limited to, a cellular response of a cell not contacted with the compound. The term “incubating” includes conditions which allow contact between the test compound and the cell of interest.

Modulation of intracellular calcium mobilization may be accomplished by inhibiting or potentiating the release of calcium from intracellular stores. For example, a compound increases calcium mobilization by increasing the release of calcium from intracellular stores and a compound decreases calcium mobilization by inhibiting the release of calcium from intracellular stores. Those of skill in the art will understand that calcium mobilization depends on the cell type and on the ligand activating the intracellular pathway. Methods of measuring free calcium flux are well known in the art, for example, imaging methodology using calcium-sensitive dyes such as fura-2 and the like may be used.

Compounds that modulate a cellular response can include peptides, peptidomimetics, polypeptides, pharmaceuticals, chemical compounds and biological agents, for example. Antibodies, neurotropic agents, anti-epileptic compounds and combinatorial compound libraries can also be tested using the method of the invention. One class of organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups.

The test agent may also be a combinatorial library for screening a plurality of compounds. Compounds such as peptides identified in the method of the invention can be further cloned, sequenced, and the like, either in solution of after binding to a solid support, by any method usually applied to the isolation of a specific DNA sequence Molecular techniques for DNA analysis (Landegren et al., Science 242:229-237, 1988) and cloning have been reviewed (Sambrook et al., Molecular Cloning: a Laboratory Manual, 2nd Ed.; Cold Spring Harbor Laboratory Press, Plainview, N.Y., 1998, herein incorporated by reference).

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

A variety of other agents may be included in the screening assay. These include agents like salts, neutral proteins, e.g., albumin, detergents, etc. that are used to facilitate optimal protein-protein binding and/or reduce nonspecific or background interactions. Reagents that improve the efficiency of the assay, such as protease inhibitors, nuclease inhibitors, antimicrobial agents, and the like may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 10 h will be sufficient.

PCBP polypeptide or fragments thereof of the invention may also be used to screen for agents which are capable of inhibiting or blocking or accelerate binding of a PCBP ligand protein, e.g., presenilin or Crk to PCBP, and thus may act as inhibitors or stimulators of PCBP binding and/or PCBP activity. Binding assays using a desired binding protein, immobilized or not, are well known in the art and may be used for this purpose using a PCBP ligand protein such as presenilin. Appropriate screening assays may be cell-based or cell-free. Alternatively, purified protein based screening assays may be used to identify such agents. For example, PCBP ligand protein may be immobilized in purified form on a carrier and binding to purified PCBP may be measured in the presence and in the absence of potential inhibiting agents. A suitable binding assay may alternatively employ purified PCBP immobilized on a carrier, with a soluble form of a PCBP ligand protein of the invention. Any PCBP ligand protein may be used in the screening assays described above.

In such a screening assay, a first binding mixture is formed by combining PCBP and PCBP ligand protein, and the amount of binding in the first binding mixture is measured. A second binding mixture is also formed by combining PCBP, PCBP ligand protein, and the compound or agent to be screened, and the amount of binding in the second binding mixture is measured. The amounts of binding in the first and second binding mixtures are compared, for example, by performing a calculation of binding in the first mixture divided by the binding in the second mixture. A compound or agent is considered to be capable of inhibiting PCBP binding, for example, if a decrease in binding in the second binding mixture as compared to the first binding mixture is observed. The formulation and optimization of binding mixtures is within the level of skill in the art. Such binding mixtures may also contain buffers and salts necessary to enhance or to optimize binding, and additional control assays may be included in the screening assay of the invention. It should be understood that fragments of PCBP can also be used in such binding/screening assays, particularly fragments containing the presenilin or Crk binding domain Such binding assays are also useful for identifying PCBP binding domains.

Alternatively, appropriate screening assays may be cell based. For example, the binding or interaction between a PCBP binding protein, e.g., presenilin and the PCBP can be measured in yeast.

Compounds found to reduce, preferably by at least about 10%, more preferably greater than about 50% or more, the binding activity of PCBP ligand protein to PCBP may thus be identified and then secondarily screened in other binding assays, including in vivo assays. By these means compounds having inhibitory activity for PCBP binding which may be suitable as antagonists may be identified.

In another embodiment of the invention, PCBP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between PCBP and the agent being tested may be measured.

Another technique for drug screening provides for high throughput screening of compounds having suitable binding affinity to the protein of interest. In this method, large numbers of different small test compounds are synthesized on a solid substrate, such as plastic pins or some other surface. The test compounds are reacted with PCBP, or fragments thereof, and washed. Bound PCBP is then detected by methods well known in the art. Purified PCBP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding PCBP specifically compete with a test compound for binding PCBP. In this manner, antibodies can be used to detect the presence of any peptide which shares one or more antigenic determinants with PCBP.

A method is further provided for treating a subject with a disorder associated with intracellular calcium mobilization or having or at risk of having a neurodegenerative disorder. The method includes administering to the subject a therapeutically effective amount of a compound that modulates Presenilin/Crk binding polypeptide activity or expression (level).

The term “modulate,” as it appears herein, refers to a change in the activity or level of PCBP. For example, modulation may cause an increase or a decrease in protein activity, binding characteristics, expression or any other biological, functional, or immunological properties of PCBP. The term “modulate” envisions the increased expression of PCBP polynucleotide when PCBP is under-expressed. Alternatively, when a disorder is associated with under-expression of PCBP polypeptide, a sense polynucleotide sequence (the DNA coding strand) encoding PCBP polypeptide, or 5′ regulatory nucleotide sequences (i.e., promoter) of PCBP in operable linkage with PCBP polynucleotide can be introduced into a cell. Therefore, the present invention also provides gene therapy for the treatment of cell proliferative disorders which are mediated by PCBP. Such therapy would achieve its therapeutic effect by introduction of the appropriate PCBP polynucleotide which contains a PCBP structural gene (sense), into cells of subjects having the disorder. Delivery of sense PCBP polynucleotide constructs can be achieved using a recombinant expression vector such as a chimeric virus or a colloidal dispersion system.

Essentially, any disorder that is etiologically linked to a Presenilin/Crk binding polypeptide, a Presenilin polypeptide, or a Crk polypeptide could be considered susceptible to treatment with an agent that modulates Presenilin/Crk binding polypeptide activity or level. The disorder may be a neuronal cell disorder. Examples of neuronal cell disorders include but are not limited to neurodegeneration, psychiatric disorders, schizophrenia, mood disorders, disorders of extrapyramidal motor function, disorders of motor control and function, attention deficit disorders, concentration disorders, pain disorders, epilepsy, convulsive disorders, eating disorders, sleep disorders, sexual disorders, circadian disorders, drug withdrawal, drug addiction, compulsive disorders, anxiety, panic disorders, or depressive disorders. Further examples of disorders include Alzheimer's disease, Amyotrophic Lateral Sclerosis (ALS) and Parkinson's disease.

Detection of altered (decreased or increased) levels of a Presenilin/Crk binding polypeptide or altered activity can be accomplished by hybridization of nucleic acids isolated from a cell of interest with a Presenilin/Crk binding protein polynucleotide of the invention. Analysis, such as Northern Blot analysis, are utilized to quantitate expression of Presenilin/Crk binding polypeptide, such as to measure Presenilin/Crk binding polypeptide transcripts. Other standard nucleic acid detection techniques will be known to those of skill in the art. Detection of altered levels of Presenilin/Crk binding protein activity can also accomplished using assays designed to detect Presenilin/Crk binding polypeptide. For example, antibodies or peptides that specifically bind a Presenilin/Crk binding protein polypeptide can be utilized. Analyses, such as Western blot analysis, radioimmune assay or immunohistochemistry, are then used to measure Presenilin/Crk binding polypeptide concentration qualitatively or quantitatively. Binding assays as described herein can be performed to assess PCBP binding activity.

In accordance with another aspect of the invention, the proteins of the invention can be used as starting points for rational drug design to provide ligands, therapeutic drugs or other types of small chemical molecules. Alternatively, small molecules or other compounds identified by the above-described screening assays may serve as ‘lead compounds’ in rational drug design. A compound useful for modulation of a Presenilin/Crk binding polypeptide activity may be an agonist or antagonist of Presenilin/Crk binding polypeptide activity. The terms “agonist” and “antagonist” are meant to include compounds that accelerate or inhibit Presenilin/Crk binding protein activity.

In another series of embodiments, the present invention provides methods and pharmaceutical preparations for use in the treatment of PCBP-associated diseases such as AD. These methods and pharmaceuticals are based upon administration of normal PCBPs, gene therapy with normal PCBP genes to compensate for or replace the mutant genes, gene therapy based upon antisense sequences to mutant PCBP genes or which “knock-out” the mutant genes, gene therapy based upon sequences which encode a protein which blocks or corrects the deleterious effects of PCBP mutants, immunotherapy based upon antibodies to normal and/or mutant PCBPs, or small molecules (drugs) which alter PCBP expression, block or enhance abnormal interactions between mutant forms of PCBP and other proteins or ligands (i.e., Presenilin or Crk), or which otherwise block the aberrant function of mutant PCBPs by altering the structure of the mutant proteins, by enhancing their metabolic clearance, or by inhibiting their function, and the like.

As used herein, “treating” refers to inhibiting or arresting the development of a disease, disorder or condition and/or causing the reduction, remission, or regression of a disease, disorder or condition. Those of skill in the art will understand that various methodologies and assays may be used to assess the development of a disease, disorder or condition, and similarly, various methodologies and assays may be used to assess the reduction, remission or regression of a disease, disorder or condition.

As used herein, “administering” refers to means for providing a therapeutically effective amount of a compound to a subject, using oral, sublingual intravenous, subcutaneous, transcutaneous, intramuscular, intracutaneous, intrathecal, epidural, intraocular, intracranial, inhalation, rectal, vaginal, and the like administration. Administration in the form of creams, lotions, tablets, capsules, pellets, dispersible powders, granules, suppositories, syrups, elixirs, lozenges, injectable solutions, sterile aqueous or non-aqueous solutions, suspensions or emulsions, patches, and the like, is also contemplated. The active ingredients may be compounded with non-toxic, pharmaceutically acceptable carriers including glucose, lactose, gum acacia, gelatin, mannitol, starch paste, magnesium trisilicate, talc, corn starch, keratin, colloidal silica, potato starch, urea, dextrans, and the like.

The preferred route of administration will vary with the clinical indication. Some variation in dosage will necessarily occur depending upon the condition of the patient being treated, and the physician will, in any event, determine the appropriate dose for the individual patient. The effective amount of compound per unit dose depends, among other things, on the body weight, physiology, and chosen inoculation regimen. A unit dose of compound refers to the weight of compound without the weight of carrier (when carrier is used).

Treatment can include modulation of Presenilin/Crk binding polypeptide activity or level by administration of a therapeutically effective amount of a compound that modulates Presenilin/Crk binding polypeptide or Presenilin/Crk binding polypeptide activity. The term “modulate” with respect to level of protein envisions the suppression of Presenilin/Crk binding protein-activity or expression when Presenilin/Crk binding protein is overexpressed or has an increased activity as compared to control. The term “modulate” also includes the augmentation of the expression of Presenilin/Crk binding polypeptide when it is underexpressed or has a decreased activity as compared to a control. When a disorder is associated with underexpression or expression of a mutant Presenilin/Crk binding polypeptide, a sense polynucleotide sequence (the DNA coding strand) or Presenilin/Crk binding polypeptide can be introduced into the cell. When a disorder is associated with overexpression of a Presenilin/Crk binding polypeptide, treatment may, for example, inhibit the interaction of the SH3 domain with its target protein, may decrease the avidity of this interaction by means of allosteric effects, may block the binding activity of a Presenilin/Crk binding polypeptide with a Presenilin polypeptide or influence other functional properties of Presenilin/Crk binding polypeptides.

Candidate agents include nucleic acids encoding a Presenilin/Crk binding polypeptide, or that interfere with expression of Presenilin/Crk binding polypeptide, such as an antisense nucleic acid, ribozymes, and the like. Candidate agents also encompass numerous chemical classes wherein the agent modulates Presenilin/Crk binding protein expression or activity.

Where a disorder is associated with the decreased expression of a Presenilin/Crk binding polypeptide, nucleic acid sequences that promote with the expression of the Presenilin/Crk binding polypeptide can be used. In this manner, the interacting of Presenilin/Crk binding polypeptides and Presenilin polypeptides can be enhanced. Where a disorder is associated with the increased expression of a Presenilin/Crk binding polypeptide, nucleic acid sequences that inhibit the expression of the Presenilin/Crk binding polypeptide can be used. This approach also utilizes, for example, antisense nucleic acid, ribozymes, or triplex agents to block transcription or translation of Presenilin/Crk binding protein mRNA, either by masking that mRNA with an antisense nucleic acid or triplex agent, or by cleaving it with a ribozyme in disorders associated with increased Presenilin/Crk binding polypeptide. Alternatively, a dominant negative form of Presenilin/Crk binding polypeptide could be administered.

A method is also provided for identifying a compound that stimulates a Presenilin/Crk binding polypeptide activity. The method includes incubating the compound and a cell expressing a Presenilin/Crk binding polypeptide under conditions sufficient to permit the compound to interact with the cell and measuring the effect of the compound on the Presenilin Crk binding polypeptide activity. One exemplary Presenilin/Crk binding polypeptide activity is modulation of intracellular calcium concentration. The modulation may be a decrease in intracellular calcium concentration or an increase in intracellular calcium concentration.

A further embodiment of the invention provides a kit useful for the diagnosis of a subject having or at risk of having a disorder of the nervous system, e.g., Alzheimer's disease (AD). The kit contains carrier means having at least one container containing a reagent that interacts with a Presenilin/Crk binding polypeptide thereby allowing the level or activity of the Presenilin/Crk binding polypeptide to be determined. Examples of reagents that interact with PCBP include antibodies or peptides of the invention (e.g., SEQ ID NO:3). The “interacting agent” or “probes” can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probes or will be able to ascertain such, using routine experimentation.

Optionally, one of the container means may comprise a probe which is or can be detectably labeled. Such probe may be an antibody or nucleotide specific for a target protein or a target nucleic acid, respectively, wherein the target is indicative, or correlates with, the presence or level of PCBP of the invention. Where the kit utilizes nucleic acid hybridization to detect the target nucleic acid, the kit may also have containers containing nucleotide(s) for amplification of the target nucleic acid sequence and/or a container comprising a reporter-means, such as a biotin-binging protein, such as avidin or streptavidin, bound to a reporter molecule, such as an enzymatic, fluorescent, or radionucleotide label.

In another series of embodiments, the present invention provides transgenic animal models for AD and other diseases or disorders associated with mutations in the PCBP genes. The animal may be essentially any amphibian, reptile, fish, mammal, and the like. Preferably, the transgenic animal is mammalian including rats, mice, hamsters, guinea pigs, rabbits, dogs, cats, goats, sheep, pigs, and non-human primates. In addition, invertebrate models, including nematodes and insects, may be used for certain applications. The animal models are produced by standard transgenic methods including microinjection, transfection, or by other forms of transformation of embryonic stem cells, zygotes, gametes, and germ line cells with vectors including genomic or cDNA fragments, minigenes, homologous recombination vectors, viral insertion vectors and the like. Suitable vectors include vaccinia virus, adenovirus, adeno associated virus, retrovirus, liposome transport, neuraltropic viruses, Herpes simplex virus, and the like. The animal models may include transgenic sequences comprising or derived from PCBPs, including normal and mutant sequences, intronic, exonic and untranslated sequences, and sequences encoding subsets of PCBPs such as functional domains. The major types of animal models provided include: (1) Animals in which a normal human PCBP gene has been recombinantly introduced into the genome of the animal as an additional gene, under the regulation of either an exogenous or an endogenous promoter element, and as either a minigene or a large genomic fragment; in which a normal human PCBP gene has been recombinantly substituted for one or both copies of the animal's homologous PCBP gene by homologous recombination or gene targeting; and/or in which one or both copies of one of the animal's homologous PCBP genes have been recombinantly “humanized” by the partial substitution of sequences encoding the human homologue by homologous recombination or gene targeting. (2) Animals in which a mutant human PCBP gene has been recombinantly introduced into the genome of the animal as an additional gene, under the regulation of either an exogenous or an endogenous promoter element, and as either a minigene or a large genomic fragment; in which a mutant human PCBP gene has been recombinantly substituted for one or both copies of the animal's homologous PCBP gene by homologous recombination or gene targeting; and/or in which one or both copies of one of the animal's homologous PCBP genes have been recombinantly “humanized” by the partial substitution of sequences encoding a mutant human homologue by homologous recombination or gene targeting. (3) Animals in which a mutant version of one of that animal's PCBP genes has been recombinantly introduced into the genome of the animal as an additional gene, under the regulation of either an exogenous or an endogenous promoter element, and as either a minigene or a large genomic fragment; and/or in which a mutant version of one of that animal's PCBP genes has been recombinantly substituted for one or both copies of the animal's homologous PCBP gene by homologous recombination or gene targeting. (4) “Knock-out” animals in which one or both copies of one of the animal's PCBP genes that have been partially or completely deleted by homologous recombination or gene targeting, or have been inactivated by the insertion or substitution by homologous recombination or gene targeting of exogenous sequences. In preferred embodiments, a transgenic mouse model for AD has a transgene encoding a normal human PCBP, a mutant human or murine PCBP, or a humanized normal or mutant murine PCBP generated by homologous recombination or gene targeting.

In a preferred embodiment of the invention, there is provided a transgenic non-human animal having a transgene that expresses a Presenilin/Crk binding protein polynucleotide chromosomally integrated into the germ cells of the animal. Animals are referred to as “transgenic” when such animal has had a heterologous DNA sequence, or one or more additional DNA sequences normally endogenous to the animal (collectively referred to herein as “transgenes”) chromosomally integrated into the germ cells of the animal. The transgenic animal (including its progeny) will also have the transgene fortuitously integrated into the chromosomes of somatic cells.

Various methods to make the transgenic animals of the subject invention can be employed. Generally speaking, three such methods may be employed. In one such method, an embryo at the pronuclear state (a “one cell embryo”) is harvested from a female and the transgene is microinjected into the embryo, in which case the transgene will be chromosomally integrated into both the germ cells and somatic cells of the resulting mature animal. In another such method, embryonic stem cells are isolated and the transgene incorporated therein by electroporation, plasmid transfection or microinjection, followed by reintroduction of the stem cells into the embryo where they colonize and contribute to the germ line. Methods for microinjection of mammalian species is described in U.S. Pat. No. 4,873,191. In yet another such method, embryonic cells are infected with a retrovirus containing the transgene whereby the germ cells of the embryo have the transgene chromosomally integrated therein. When the animals to be made transgenic are avian, because avian fertilized ova generally go through cell division for the first twenty hours in the oviduct, microinjection into the pronucleus of the fertilized egg is problematic due to the inaccessibility of the pronucleus. Therefore, of the methods to make transgenic animals described generally above, retrovirus infection is preferred for avian species, for example as described in U.S. Pat. No. 5,162,215. If microinjection is to be used with avian species, however, a recently published procedure by Love et al. (Biotechnology, 12, January 1994) can be utilized whereby the embryo is obtained from a sacrificed hen approximately two and one-half h after the laying of the previous laid egg, the transgene is microinjected into the cytoplasm of the germinal disc and the embryo is cultured in a host shell until maturity. When the animals to be made transgenic are bovine or porcine, microinjection can be hampered by the opacity of the ova thereby making the nuclei difficult to identify by traditional differential interference-contrast microscopy. To overcome this problem, the ova can first be centrifuged to segregate the pronuclei for better visualization.

The non-human animals of the invention are murine typically (e.g., mouse). The transgenic non-human animals of the invention are produced by introducing “transgenes” into the germline of the non-human animal. Embryonal target cells at various developmental stages can be used to introduce transgenes. Different methods are used depending on the stage of development of the embryonal target cell. The zygote is the best target for microinjection. The use of zygotes as a target for gene transfer has a major advantage in that in most cases the injected DNA will be incorporated into the host gene before the first cleavage (Brinster et al., Proc. Natl. Acad. Sci. USA, 82:4438-4442, 1985). As a consequence, all cells of the transgenic non-human animal will carry the incorporated transgene. This will in general also be reflected in the efficient transmission of the transgene to offspring of the founder since 50% of the germ cells will harbor the transgene.

The term “transgenic” is used to describe an animal which includes exogenous genetic material within all of its cells. A “transgenic” animal can be produced by cross-breeding two chimeric animals which include exogenous genetic material within cells used in reproduction. Twenty-five percent of the resulting offspring will be transgenic i.e., animals which include the exogenous genetic material within all of their cells in both alleles. 50% of the resulting animals will include the exogenous genetic material within one allele and 25% will include no exogenous genetic material.

In the microinjection method useful in the practice of the subject invention, the transgene is digested and purified free from any vector DNA, e.g., by gel electrophoresis. It is preferred that the transgene include an operatively associated promoter which interacts with cellular proteins involved in transcription, ultimately resulting in constitutive expression. Promoters useful in this regard include those from cytomegalovirus (CMV), Moloney leukemia virus (MLV), and herpes virus, as well as those from the genes encoding metallothionein, skeletal actin, P-enolpyruvate carboxylase (PEPCK), phosphoglycerate (PGK), DHFR, and thymidine kinase. Promoters for viral long terminal repeats (LTRs) such as Rous Sarcoma Virus can also be employed. Constructs useful in plasmid transfection of embryonic stem cells will employ additional regulatory elements well known in the art such as enhancer elements to stimulate transcription, splice acceptors, termination and polyadenylation signals, and ribosome binding sites to permit translation.

Retroviral infection can also be used to introduce transgene into a non-human animal, as described above. The developing non-human embryo can be cultured in vitro to the blastocyst stage. During this time, the blastomeres can be targets for retro viral infection (Jaenich, R., Proc. Natl. Acad. Sci. USA, 73:1260-1264, 1976). Efficient infection of the blastomeres is obtained by enzymatic treatment to remove the zona pellucida (Hogan et al., 1986) in Manipulating the Mouse Embryo, Cold Spring Harbor Laboratory Press, Cold Spring Harbor, N.Y.). The viral vector system used to introduce the transgene is typically a replication-defective retro virus carrying the transgene (Jahner et al., Proc. Natl. Acad. Sci. USA, 82:6927-6931, 1985; Van der Putten et al., Proc. Natl. Acad. Sci. USA, 82:6148-6152, 1985). Transfection is easily and efficiently obtained by culturing the blastomeres on a monolayer of virus-producing cells (Van der Putten, supra; Stewart et al., EMBO J., 6:383-388, 1987). Alternatively, infection can be performed at a later stage. Virus or virus-producing cells can be injected into the blastocoele (D. Jahner et al., Nature, 2-98:623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retroviral infection of the midgestation embryo (D. Jahner et al., supra).

A third type of target cell for transgene introduction is the embryonal stem cell (ES). ES cells are obtained from pre-implantation embryos cultured in vitro and fused with embryos (M. J. Evans et al., Nature, 292:154-156, 1981; M. O. Bradley et al., Nature 309:255-258, 1984; Gossler et al., Proc. Natl. Acad. Sci. USA, 83:9065-9069, 1986; and Robertson et al., Nature, 322:445-448, 1986). Transgenes can be efficiently introduced into the ES cells by DNA transfection or by retro virus-mediated transduction. Such transformed ES cells can thereafter be combined with blastocysts from a nonhuman animal. The ES cells thereafter colonize the embryo and contribute to the germ line of the resulting chimeric animal. (For review see Jaenisch, R., Science, 240:1468-1474, 1988).

“Transformed” means a cell into which (or into an ancestor of which) has been introduced, by means of recombinant nucleic acid techniques, a heterologous nucleic acid molecule. “Heterologous” refers to a nucleic acid sequence that either originates from another species or is modified from either its original form or the form primarily expressed in the cell.

“Transgene” means any piece of DNA which is inserted by artifice into a cell, and becomes part of the genome of the organism (i.e., either stably integrated or as a stable extrachromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence which is transcribed into DNA and then incorporated into the genome. The transgenes of the invention include DNA sequences which encode Presenilin/Crk binding polypeptide-sense and antisense polynucleotides, which may be expressed in a transgenic non-human animal. The term “transgenic” as used herein additionally includes any organism whose genome has been altered by in vitro manipulation of the early embryo or fertilized egg or by any transgenic technology to induce a specific gene knockout. As used herein, the term “transgenic” includes any transgenic technology familiar to those in the art which can produce an organism carrying an introduced transgene or one in which an endogenous gene has been rendered non-functional or “knocked out.”

Yet another aspect of the invention pertains to a method of treating a patient having or at risk of having a disorder associated with a decreased level of a Presenilin/Crk binding polypeptide. The method includes introducing into the patient a polynucleotide encoding the Presenilin/Crk binding polypeptide operatively linked to a regulatory sequence (see Anderson, Nature, 392:25-30 (1998)).

One approach for in vivo introduction of nucleic acid encoding one of the subject proteins into a patient is by use of a viral vector containing nucleic acid, e.g., a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood, 76:271). Thus, recombinant retrovirus can be constructed in which part of the retroviral coding sequence (gag, pol, env) has been replaced by nucleic acid encoding a Presenilin/Crk binding polypeptide, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.), Greene Publishing Associates (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example, Eglitis et al. (1985) Science, 230:1395-1398; Danos and Mulligan (1988) Proc. Natl. Acad. Sci. USA, 85:6460-6464; Wilson et al. (1988) Proc. Natl. Acad. Sci. USA. 85:3014-3018; Armentano et al. (1990) Proc. Natl. Acad. Sci. USA, 87:6141-6145; Huber et al. (1991) Proc. Natl. Acad. Sci. USA, 88:8039-8043; Ferry et al. (1991) Proc. Natl. Acad. Sci. USA, 88:8377-8381; Chowdhury et al. (1991) Science, 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA, 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In choosing retroviral vectors as a gene delivery system for the subject Presenilin/Crk binding protein genes, it is important to note that a prerequisite for the successful infection of target cells by most retroviruses, and therefore of stable introduction of the recombinant Presenilin/Crk binding protein gene, is that the target cells must be dividing. In general, this requirement will not be a hindrance to use of retroviral vectors to deliver antagonistic Presenilin/Crk binding protein gene constructs. In fact, such limitation on infection can be beneficial in circumstances wherein the tissue (e.g., nontransformed cells) surrounding the target cells does not undergo extensive cell division and is therefore refractory to infection with retroviral vectors.

Furthermore, it has been shown that it is possible to limit the infection spectrum of retroviruses and consequently of retroviral-based vectors, by modifying the viral packaging proteins on the surface of the viral particle (see, for example, PCT publications WO93/25234, WO94/06920, and WO94/11524). For instance, strategies for the modification of the infection spectrum of retroviral vectors include: coupling antibodies specific for cell surface antigens to the viral env protein (Roux et al. (1989) Proc. Natl. Acad. Sci. USA 86:9079-9083; Julan et al. (1992) J. Gen. Virol., 73:3251-3255; and Goud et al. (1983) Virology, 163:251-254); or coupling cell surface ligands to the viral env proteins (Neda et al. (1991) J. Biol. Chem., 266:14143-14146). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to limit or otherwise direct the infection to certain tissue types, and can also be used to convert an ecotropic vector into an amphotropic vector.

Moreover, use of retroviral gene delivery can be further enhanced by the use of tissue- or cell-specific transcriptional regulatory sequences which control expression of the Presenilin/Crk binding protein gene of the retroviral vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al. (1988) BioTechniques, 6:616; Rosenfeld et al. (1991) Science 252:431-434; and Rosenfeld et al. (1992) Cell, 68:143-155). Suitable adenoviral vectors derived from the adenovirus strain Ad type 5 d11324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA, 90:2812-2816) and muscle cells (Quantin et al. (1992) Proc. Natl. Acad. Sci. USA, 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol., 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell, 16:683; Berkner et al., supra; and Graham et al., in Methods in Molecular Biology, E. J. Murray, Ed. (Human, Clifton, N.J., 1991) vol. 7 pp. 109-127). Expression of the inserted Presenilin/Crk binding protein gene can be under control of, for example, the E1A promoter, the major late promoter (MLP) and associated leader sequences, the E3 promoter, or exogenously added promoter sequences.

Yet another viral vector system useful for delivery of the subject genes is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. (1992) 158:97-129). It is also one of the few viruses that may integrate its DNA into non-dividing cells, and exhibits a high frequency of stable integration (see for example, Flotte et al. (1992) Am. J. Respir. Cell. Mol. Biol., 7:349-356; Samulski et al. (1989) J. Virol., 63:3822-3828; and McLaughlin et al. (1989) J. Virol., 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol., 5:3251-3260 can be used to introduce DNA into cells. A variety of nucleic acids have been introduced into different cell types using AAV vectors (see for example, Hermonat et al. (1984) Proc. Natl. Acad. Sci. USA, 81:6466-6470; Tratschin et al. (1985) Mol. Cell. Biol. 4:2072-2081; Wondisford et al. (1988) Mol. Endocrinol., 2:32-39; Tratschin et al. (1984); J. Virol., 51:611-619; and Flotte et al. (1993) J. Biol. Chem., 268:3781-3790).

Other viral vector systems that may have application in gene therapy have been derived from herpes virus, vaccinia virus, and several RNA viruses. In particular, herpes virus vectors may provide a unique strategy for persistence of the recombinant Presenilin/Crk binding protein gene in cells of the central nervous system and ocular tissue (Pepose et al. (1994) Invest. Ophthalmol. Vis. Sci., 35:2662-2666).

In addition to viral transfer methods, such as those illustrated above, non-viral methods can also be employed to cause expression of a Presenilin/Crk binding polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject Presenilin/Crk binding protein gene by the targeted cell. Exemplary gene delivery systems of this type include liposomal-derived systems, poly-lysine conjugates, and artificial viral envelopes.

In clinical settings, the gene delivery systems can be introduced into a patient by any of a number of methods, each of which is familiar in the art. For instance, a pharmaceutical preparation of the gene delivery system can be introduced systemically, e.g., by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) Proc. Natl. Acad. Sci. USA., 91:3054-3057).

Moreover, the pharmaceutical preparation can consist essentially of the gene delivery system in an acceptable diluent, or can comprise a slow release matrix in which the gene delivery vehicle is imbedded. Alternatively, where the complete gene delivery system can be produced intact from recombinant cells, e.g., retroviral packages, the pharmaceutical preparation can comprise one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinaceous biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

By choice of monomer composition or polymerization technique, the amount of water, porosity and consequent permeability characteristics can be controlled. The selection of the shape, size, polymer, and method for implantation can be determined on an individual basis according to the disorder to be treated and the individual patient response. The generation of such implants is generally known in the art. See, for example, Concise Encyclopedia of Medical & Dental Materials, ed. by David Williams (MIT Press: Cambridge, Mass., 1990); and the Sabel et al., U.S. Pat. No. 4,883,666. In another embodiment of an implant, a source of cells producing the recombinant virus is encapsulated in implantable hollow fibers. Such fibers can be pre-spun and subsequently loaded with the viral source (Aebischer et al., U.S. Pat. No. 4,892,538; Aebischer et al., U.S. Pat. No. 5,106,627; Hoffman et al. (1990) Expt. Neurobiol., 110:39-44; Jaeger et al. (1990) Prog. Brain Res. 82:4146; and Aebischer et al. (1991) J. Biomech. Eng., 113:178-183), or can be co-extruded with a polymer which acts to form a polymeric coat about the viral packaging cells (Lim, U.S. Pat. No. 4,391,909; Sefton, U.S. Pat. No. 4,353,888; Sugamori et al. (1989) Trans. Am. Artif. Intern. Organs, 35:791-799; Sefton et al. (1987) Biotechnol. Bioeng., 29:1135-1143; and Aebischer et al. (1991) Biomaterials, 12:50-55). Again, manipulation of the polymer can be carried out to provide for optimal release of viral particles.

The invention also provides an excision knockout mouse whose genome comprises an excision of a Presenilin/Crk binding polypeptide gene. The excision is accomplished by homologous recombination and results in the mouse exhibiting a disorder of the nervous system as compared to a wild-type mouse.

The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLES

### Example 1

### Identification of a Presenilin Binding Protein Polypeptide

The yeast two-hybrid system was used to screen for cytoplasmic proteins that interact with PSs. The PSs are predominantly localized to the endoplasmic reticulum, and the major fragments exposed to the cytoplasm are thought to be the amino terminus (amino acids 1-81), the carboxy terminus (amino acids 429-468), and a large hydrophilic loop (amino acids 263-407) (Busciglio et al. (1997); Kovac et al., Nature Med. 2:224-229 (1996); Doan et al., Neuron, 17:1023-1030 (1996)). cDNA sequences encoding the amino-terminal, carboxy terminal and the large hydrophilic loop region of PS1 (amino acids 1-81, 429-468, 263-407, respectively) were inserted into the pGBT9 vector (Clontech, Palo Alto, Calif.) as a fusion to the GAL4-DNA binding domain. Yeast (Y190) were co-transformed with each of the PS1 constructs and a mouse brain cDNA library fused to the GAL4 activation domain in the pGAD424 vector (Clontech). Transformants were plated on yeast drop-out media (Clontech) contain g 20 mM 3-amino-1,2,4-triazole (3-AT), and after 10 days of incubation at 30° C., colonies were transferred to Nitropure membrane Micron Separation Inc., Westboro, MA), and the X-gal assay was performed.

One positive clone containing a 1.3 kb insert (PBPα) was obtained after screening 10⁷ independent clones using the large hydrophilic loop of PS1 as a bait sequence. No positive clones were obtained with the amino-terminus or carboxy-terminus fragments as bait.

### Example 2

### Hybridization Cloning

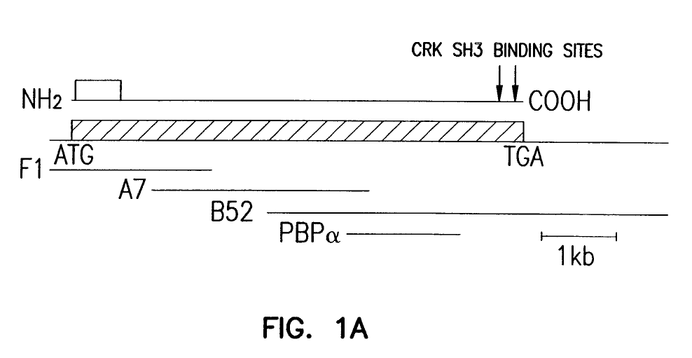

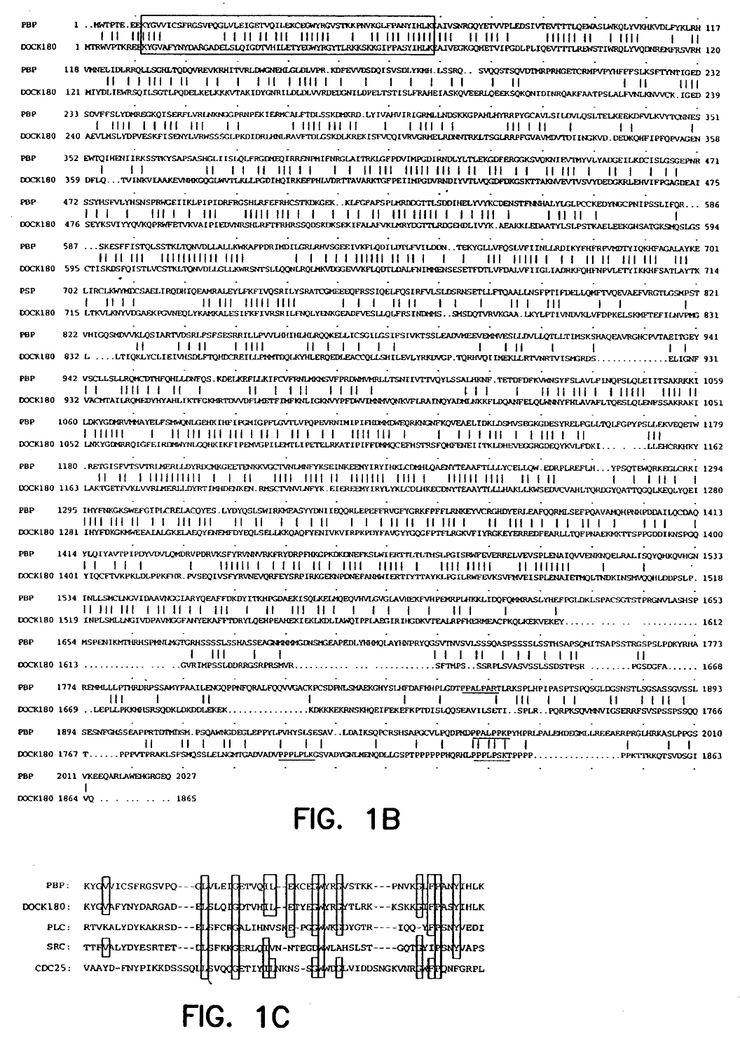

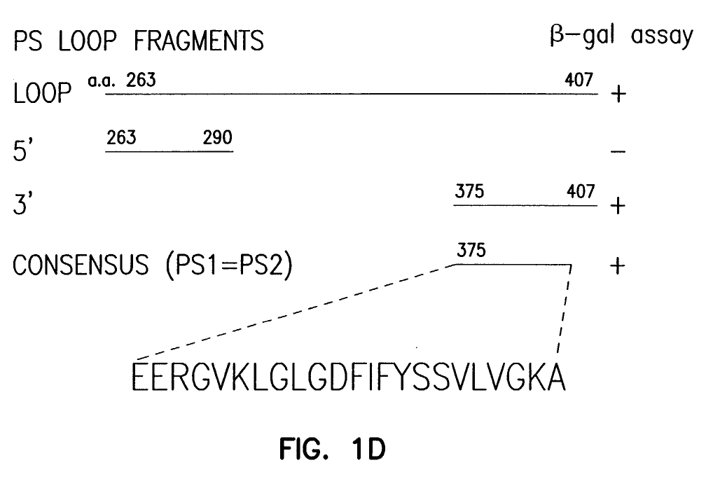

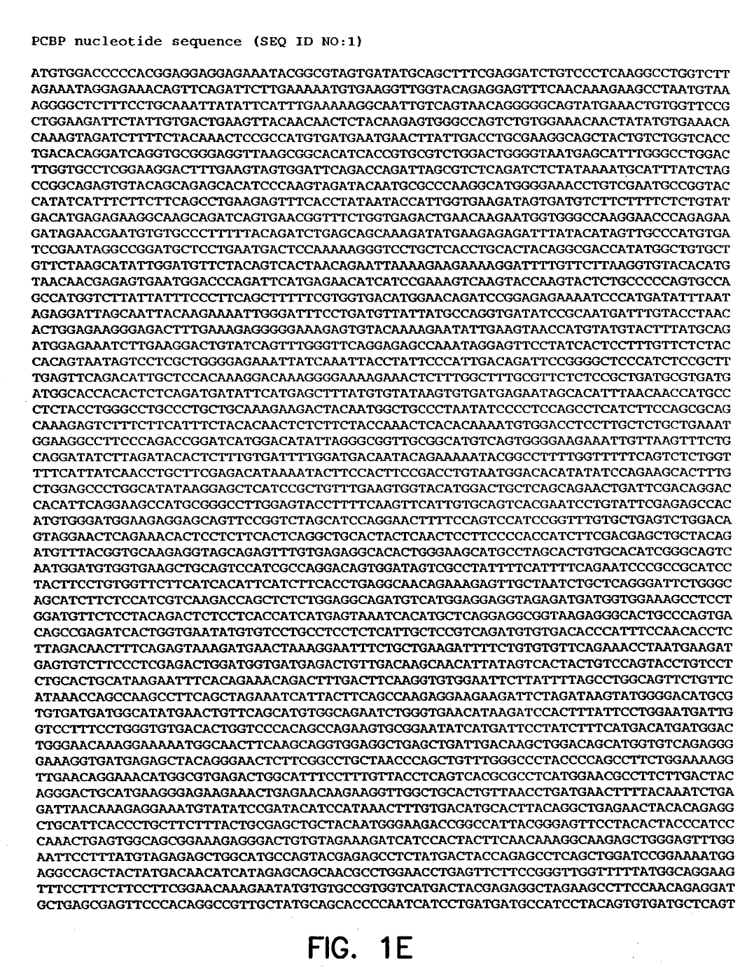

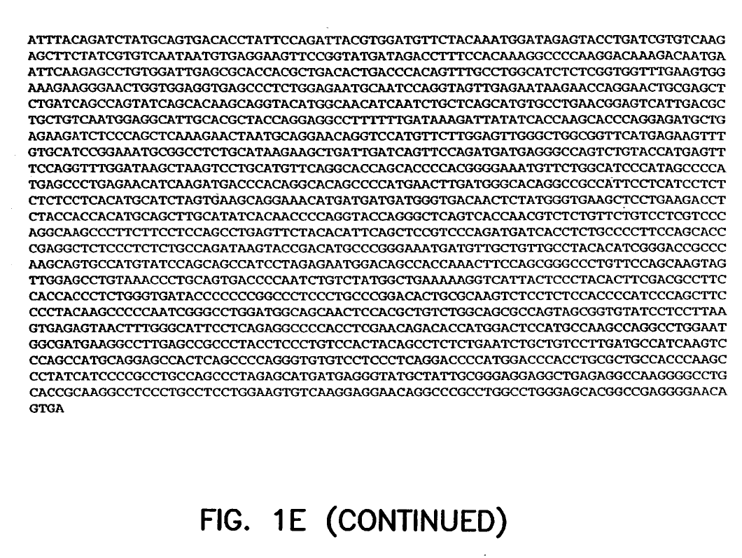

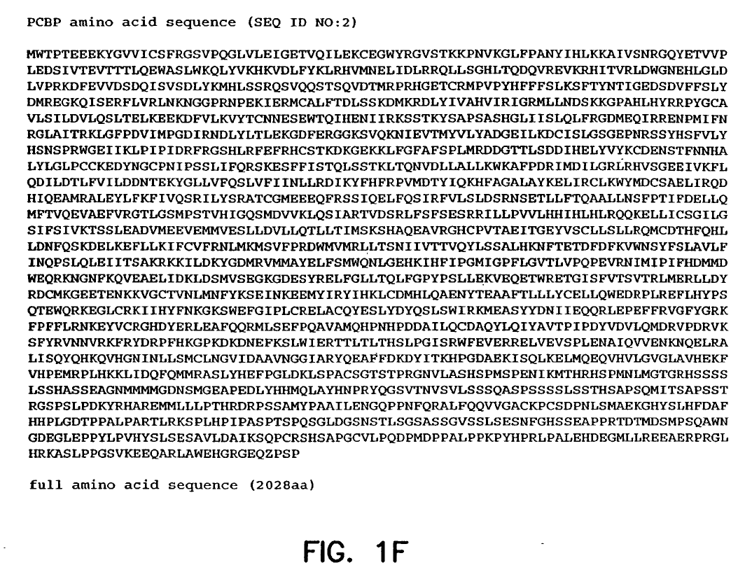

To obtain a full-length cDNA clone, a mouse brain cDNA library (Stratagene, La Jolla, Calif.) was screened by using ³²P labeled-PBPα as a hybridization probe (Kimura et al., Nature, 348:257-260 (1990)). Among 23 positive cDNA clones, three long fragments, F1 (2.0 kb), A7 (2.7 kb) and B52 (5.6 kb) were sequenced on both strands. All the nucleotide sequences within an open reading frame overlapped at least in two of these clones. These three overlapping cDNA fragments, encode a 8.6 kb message (FIG. 1a). The combined cDNA sequence contained a 6081-bp open reading frame that encodes a novel 2027 amino acid protein with a calculated molecular weight of 233 kD (FIG. 1b). We designated this protein presenilin binding protein (PBP). PBP is 40% homologous to Dock 180, which has a SH3 domain and interacts with Crk (Hasegawa et al., Molec. Cell. Biol., 16:1770-1776 (1996)). Crk is an adapter protein containing SH2 and SH3 domains that transduce tyrosine phosphorylation signals to the Ras family of proteins (Hempstead et al., Molec. Cell. Biol., 14:1964-1971 (1994)). A region in the amino terminus of PBP which fulfills the criteria for a SH3 domain has 53% homology to Dock 180 (FIG. 1c). This region is also homologous to other SH3 domains, including those in phospholipase C, Src and CDC25 (FIG. 1c). PBP has two consensus sequences, PPxLPxK(R) (SEQ ID NO:4), for the binding of the CRK SH3-binding domain in its carboxyl terminus (FIG. 1b). Finally, a partial sequence of the human counterpart of PBP has been determined (Nagase et al., DNA Res., 4:141-150 (1997)), and this maps to human chromosome 3q14.3-21.3. A putative locus for late-onset AD has also been mapped to chromosome 3 (Tanzi et al., Neurobiol. Dis., 3:159-168 (1996)).

### Example 3

### Determination of Protein Interactions

To determine the minimal sequence of the large hydrophilic loop of PS that is required for the interaction with PBP, three deletion fragments of the loop were inserted into pGBT9 and the interaction with PBP was examined using the yeast two-hybrid system. Two fragments designated 3′ and ‘consensus’ (amino acids 375-408 and 375-397, respectively) exhibit an interaction with PBP, while a fragment designated 5′ (amino acids 263-290) does not show any interaction with PBP. The fragment ‘consensus’ encodes an amino acid sequence (EERGVKLGLGDFIFYSVLVGKA) (SEQ ID NO:3) that is conserved between PS1 and PS2 (FIG. 1d). These observations suggest that PBP interacts with PSs at the 22 amino acid consensus sequence.

To confirm the yeast two-hybrid data, the interaction between PBP and PSs was assayed in mammalian cells by immunoprecipitation. Human kidney 293-T cells, and monkey kidney COS-7 cells were cultured in Dulbecco's modified Eagle's medium supplemented with 10% fetal calf serum at 37° C. in a 10% CO₂ atmosphere. A nine-amino acid TAG epitope (YPYDVPDYA) (SEQ ID NO:6) was incorporated in-frame at the 3′ end of the PBP cDNA (PBP-TAG), and an eight amino acid FLAG epitope (DYKDDDDI) (SEQ ID NO:7) was attached in frame at the 5′ end of the large hydrophilic loop of PS1 cDNA and at the 3′ end of full length PS1 cDNA as described previously (Kimura, H., Proc. Natl. Acad. Sci. (USA), 90:2165-2169 (1993)). The resulting fragments were inserted into the pCIS2 expression vector (Genentech, San Francisco, Calif.) for transient or constitutive expression.

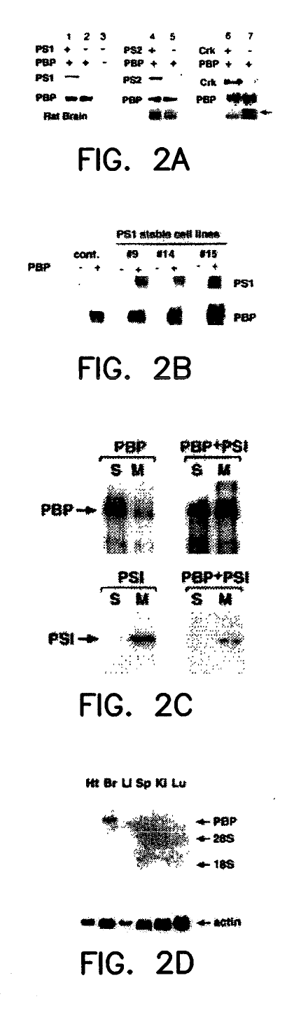

PBP-TAG was co-transfected into 293T human kidney cells with an expression plasmid containing cDNA encoding the large hydrophilic loop of PS1 and PS2 attached to the 8 amino acid epitope FLAG (PS1-FLAG) at the 5′-terminus. Forty-eight hrs after transfection, cells were lysed and proteins immunoprecipitated with a mouse monoclonal antibody, 12CA5 (anti-TAG). The immunoprecipitates were run on SDS acrylamide gels and immunoblotted with either anti PBP-TAG or anti PS1-Flag. FIG. 2a shows that PBP interacts with the large hydrophilic loops of PS1 and PS2. Dock 180, which is related to PBP, did not show any interaction with PS1, nor did it functionally interact with PS1 in the yeast two-hybrid assay. In addition, in the yeast two-hybrid system, productive interactions did not occur between PBP and a FAD mutant of PS1 (L392V). These plasmids were co-transfected into the cells using transfectamine for 293-T cells and the DEAE-dextran method for COS cells. Membrane and soluble fractions were obtained by homogenizing cells in TBS, removal of nuclei, and centrifugation at 100,000×g for 1 hr to pellet the membranes.

To examine the interaction of PBP with the fill length PS molecule, three different clonal 293T cell lines which stably express full length PS1 were transfected with PBP-TAG and the ability of PS1 to bind to PBP assayed by immunoprecipitation. Forty-eight hrs after transfection, cells were lysed and proteins immunoprecipitated with 12CA5 (anti-TAG), which recognizes PBP-TAG (anti-PBP-TAG). The immunoprecipitates were subjected to polyacrylamide gel electrophoresis, transferred to Immobilon-P, and reacted with anti-TAG or an anti-serum against PS1. These cell lines express full length PBP, and the data in FIG. 2b shows that PBP (MW 233,000) interacts with full length PS1 (MW 43,000) as assayed by co-immunoprecipitation.

Since PBP has two consensus sequences for Crk binding, the interaction between PBP and Crk was also examined. The expression plasmid containing PBP cDNA attached to TAG and a plasmid containing Crk cDNA were co-transfected into 293T cells. Forty-eight hrs later, cells were lysed and proteins immunoprecipitated with anti-PBP-TAG and run on SDS acrylamide gels. Western blot analysis with an antibody against Crk shows that PBP co-precipitates with Crk (FIG. 2a, lanes 6 and 7).

For immunoprecipitation of the large hydrophilic loop of PSs or Crk with PBP, cells were lysed in RIPA buffer (100 mM NaCl, 0.5% NP-40, 0.5% DOC, 0.1% SDS, 50 mM Tris, pH 7.5, 1 mM PMSF and 1 μg/ml aprotinin, 1 μg/l leupeptin) on ice for 30 min. For immunoprecipitation of full length PS1 with PBP, RIPA buffer with 1% NP40 but without DOC and SDS was used. Cell lysates were spun for 10 min at 10,000 g. The resulting supernatants were preadsorbed with anti-mouse IgG-agarose (Sigma) for 4 hours at 4° C. After centrifugation, antibody 12CA5 and anti-mouse IgG-agarose were added at 4° C. overnight. After incubation with anti-mouse IgG-agarose, immunoprecipitates were subjected to SDS-polyacrylamide gel electrophoresis and transferred to Immobilon (Millipore, Bedford, Mass.). Western blot analysis with anti-FLAG and 12CA5 monoclonal antibodies or an antibody against Crk was then performed. The bound antibodies were detected by the ECL-plus chemiluminescence system (Amersham, Arlington Heights, Ill.).

### Example 4

### Cellular Localization

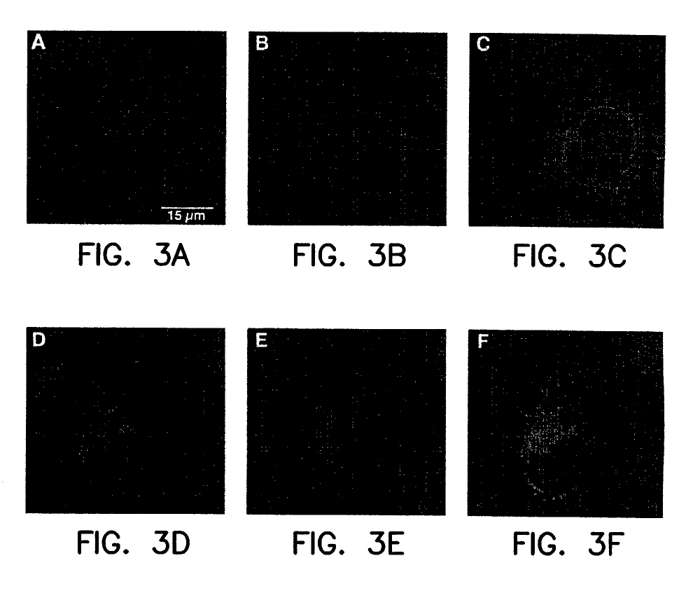

Since PS is localized to the endoplasmic reticulum (ER) (Kovac et al. (1996); Doan et al. (1996)) and PBP interacts with PS, the cellular localization of PBP was examined by immunohistochemistry using a deconvolution microscope. Expression plasmids containing full length cDNA for PBP-TAG, full length PS1 or pairs of these molecules were transfected into COS cells. Fifty hrs after the transfection, cells were fixed and stained with an anti-PBP-TAG antibody and the antibody against PS1. PBP alone diffusely localizes in the cytoplasm (FIGS. 3A, B), while PS1 alone has an apparent ER localization (FIG. 3C) consistent with previous results (Kovac et al. (1996); Doan et al. (1996)). However, cells transfected with both PS1 and PBP show patches of intense PBP-PS1 colocalization (FIGS. 3D, E, F) consistent with a PS1-dependent redistribution of PBP from a predominantly cytoplasmic to a more discrete organelle-like compartment. PS1 expressing the L392V mutation does, however, immune precipitate with PBP and colocalize with PBP in overexpressing cells.

To confirm the results of the immunohistochemistry data, cells were fractionated into soluble and membrane fractions, and Western blot analysis was performed. When PBP or PS1 alone is expressed, PBP is found predominantly in the soluble fraction and PS1 in the membrane fraction (FIG. 2c). Co-expression of PBP and PS1 causes a shift in the localization of PBP to the membrane fraction (FIG. 2c). This shift was not observed in cells transfected with Dock 180 plus PBP. These observations are consistent with the microscopic data and suggest that cytoplasmic PBP becomes associated with membranes in the presence of excess PS1.

Xenopus oocytes contain a plasma membrane CF channel which is activated by increases in intracellular Ca²⁺, and intracellular Ca²⁺ release is activated exclusively by inositol 1,4,5-triphosphate (IP₃) Miledi & Parker, J. Physiol., 415:189-210 (1989)). Lysophosphatidic acid (LPA) increases the Ca²⁺-activated Cl⁻ current in oocytes expressing PS1 relative to control oocytes (Leissring et al. (1999)). Since PBP can associate with PS1, the effect of the interaction of PBP with PS1 on the activation of Ca²⁺-activated Cl⁻ current induced by serum (Miledi & Parker (1989)) was examined with Xenopus oocytes. Although PBP alone slightly decreases the Ca²⁺-activated Cl⁻ current, co-expression of PBP with PS1 significantly suppresses the current (difference between PS1 and PS1+PBP, P<0.05 by the Student t-test, FIG. 4). The PS1-loop mutation L293V, which does not productively bind to PBP in the yeast two-hybrid system, does not suppress the Cl⁻ current in the presence of PBP. These observations suggest that intracellular Ca²⁺ mobilization is regulated by PBP in the presence of PS1, and further substantiate the interaction between PS1 and PBP. Injection of cRNAs into Xenopus oocytes and the electrophysiological recordings were done as described previously (Kimura & Schubert, Proc. Natl. Acad. Sci. (USA), 90:7508-7512 (1993)).

### Example 5

### Tissue Localization

To determine the tissue localization of PBP, Northern blot and in situ hybridization experiments were done. Northern blot analysis was performed with poly(A⁺) RNA extracted from adult mouse brain, heart, liver, spleen and kidney. Poly(A⁺) RNA was extracted from adult mouse brain, heart, liver, spleen and kidney by using FastTrack kit (Invitrogen, Carlsbad, Calif.). Six μg of each poly(A⁺) RNA was run on 1% formaldehyde agarose gels, transferred to Nylon membranes (Hybond N, Amersham, Arlington, Ill.), and probed with a ³²P-labeled 2.7 kb PBP cDNA fragment, A7 as described previously (Kimura et al. (1990)). A single transcript of 8.6 kb was identified only in the brain (FIG. 2d). No detectable mRNA was seen in heart, liver, spleen and kidney.

To determine which regions of the brain express PBP mRNA, in situ hybridization was performed. A cRNA probe was produced from a 2.7 kb cDNA fragment of the clone A7 subcloned into pBluescript SK(−) vector (Stratagene, La Jolla, Calif.) and linearized with Bam HI. Radioactive cRNA was synthesized from 250 ng of linearized plasmid with T7 polymerase. A sense probe was used as a control in some adjacent sections. Prior to hybridization, tissue sections mounted on gelatin and poly-L-lysine-coated slides were dried under a vacuum overnight, fixed (4% paraformaldehyde for 30 min), digested by proteinase K and acetylated. The clone A7 cRNA probe was applied to each slice in hybridization mixture (10⁷ cpm/ml). Hybridization was performed overnight at 60° C. The sections were digested with RNase A, washed, and dipped in NTB2 nuclear emulsion (Eastman Kodak, Rochester, N.Y.; diluted 1:1 with distilled water). Slides were exposed for 10-14 days.

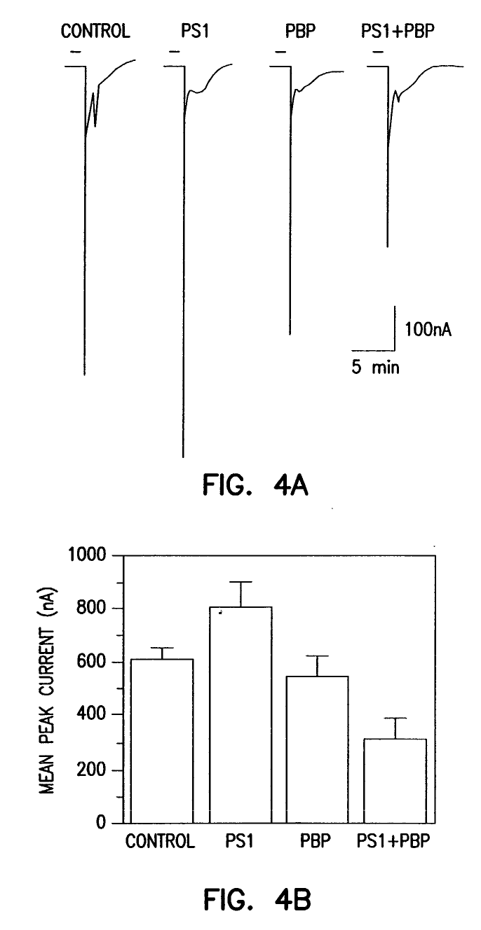

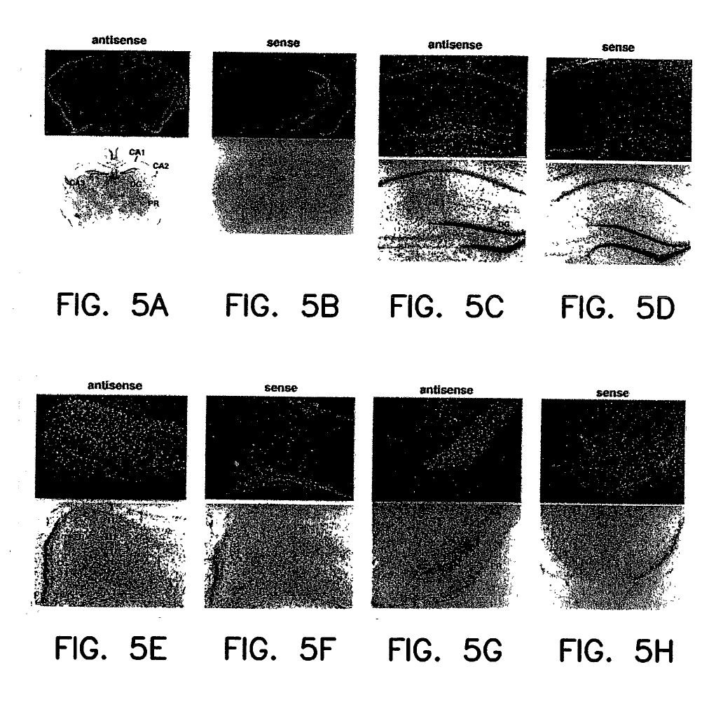

The PBP mRNA is highly localized to the cerebral cortex and hippocampus (FIG. 5), and virtually absent in other tissues except for spinal cord. In the cerebral cortex, PBP mRNA is found within the intermediate and deep layers, while only a weak signal is detected in superficial layer I (FIGS. 5a & e). PBP mRNA is also abundant in the piriform cortex (FIGS. 5a & g). Within the hippocampus, PBP mRNA is found in the pyramidal neurons of the CA1, CA2 and CA3 regions, and the dentate gyrus (FIGS. 5a & c). Signal is not detected with a sense probe (FIGS. 5b, d, f, h), indicating the specificity of the antisense probe.

### Example 6

### Localization of PBP in AD Brain

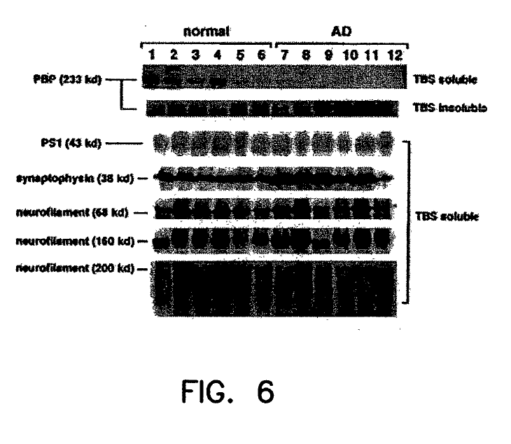

Since the altered function of PS may account for a fundamental process in AD, it is important to know the localization of PBP in AD brain. Brains of six sporadic cases that were clinically and pathologically diagnosed with AD and six brains of age-matched normal individuals were used to examine the localization of PBP. Tris-buffered saline (TBS)-soluble and insoluble fractions were prepared from the brain tissue and subjected to western blot analysis with an antibody against PBP. PBP in the TBS-soluble fraction of the AD brain is very much reduced relative to PBP in the TBS-soluble fraction of normal brain (FIG. 6). In contrast, PBP is found in the TBS-insoluble fraction of both control and AD brain, although in reduced amounts in two of the AD samples. Immunoblotting with antisera against PS1, synaptophysin, and 3 neurofilament subunits shows that there is no detectable degradation in the samples of AD brain compared with the brain samples from normal individuals (FIG. 6).

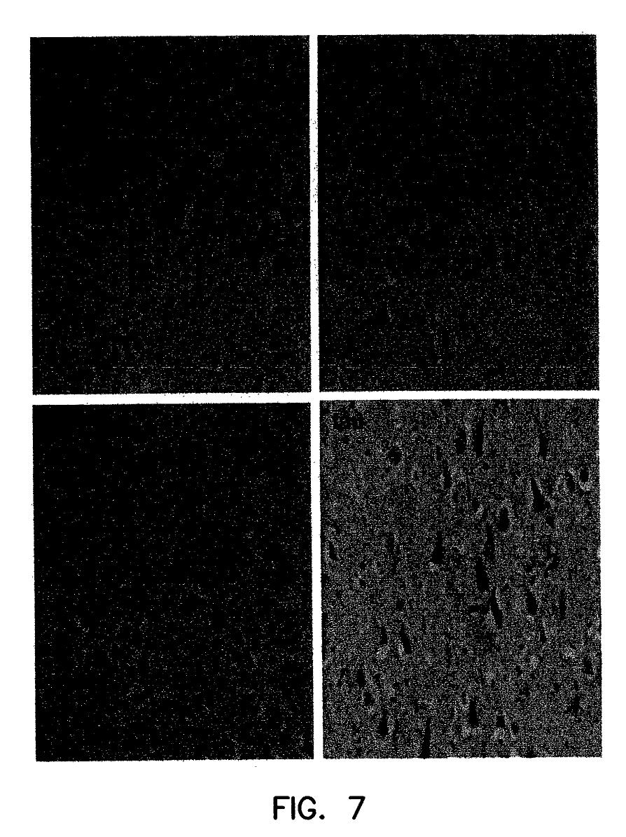

To define the cellular localization of PBP in AD brain, hippocampal slices were stained with three independent antibodies against PBP (against amino acid residues 250-270, 1973-1987 and 2012-2027), and compared to staining with antibody C5 against tau (Yoshida et al., J. Biol. Chem., 273:9761-9768 (1998)). Staining with all three PBP antibodies shows that PBP is localized to the dystrophic large neurons that stain with anti-tau (FIG. 7). To confirm these results, hippocampal slices of AD brains were double stained with anti-PBP and anti-tau. PBP and tau frequently localized to the same dystrophic neurons. Thus, PBP is associated with the large pyramidal neurons that bear neurofibrillary tangles in sporadic AD brain. Dystrophic neurons are largely localized to the second and third layer of the frontal and piriform cortices of transgenic mice expressing PS1 mutations (Chui et al., Nature Med., 5:560-564 (1999)), areas where PBP is highly expressed (FIG. 5). Another component of dystrophic neurons is hyperphosphorylated tau, a major component of neurofibrillary tangles (Bancher et al., Acta Neuropathol. (Berl), 74:39-46 (1987)). Since PBP has several structural homologies with proteins which mediate protein phosphorylation, such as Dock 180, the possibility also exists that PBP is involved in tau phosphorylation.

Immunocytochemical staining was performed using paraffin-embedded sections (5 μm). Each section was blocked with 10% calf serum in phosphate-buffered saline (PBS) and immunostained with antibodies, followed by incubation with mixture of FITC-conjugated anti-rabbit IgG and Rhodamine-conjugated anti-mouse IgG antibodies. For preparation of anti-PBP antibody, the peptides were conjugated with keyhole limpet hemocyanin (KLH). New Zealand rabbits were immunized and antibody titrated as described previously (Yoshida et al. (1998)). The resultant antisera reacted with the 233 kD PBP protein in transfected cells. Anti-PS1-loop domain antibody was a gift from Dr. Thinakaran and anti-tau from Dr. Ihara. Anti-synaptophysin was purchased from Boebringer Mannheim and anti-neurofilament from Sigma. TBA soluble and insoluble fractions were obtained by homogenizing cortical tissue in TBS, followed by centrifugation at 100,000×g for 1 hr to separate the soluble and the particulate fractions.

### Example 7

### Presenilin Binding Protein Regulates the Degradation of β-Amyloid Precursor Protein

Introduction

Presenilin (PS) binding protein, PBP, is a 240,000 MW protein which interacts with PS1 and PS2, is localized to brain areas involved in Alzheimer's disease (AD) pathology and is lost from the soluble fraction of sporadic AD brains (Kashiwa et al. (2000) J. Neurochem, 75: 109-116). PS1 has been associated with γ-secretase activity (Fraser et al. (2000) Biochem. Biophys. Acta, 1502: 1-15; Sherrington et al. (1995) Nature, 375 754-760). It is now demonstrated that expression of PBP decreases APP secretion, which is further decreased by elevated PS1 expression. This effect of PBP is due to the acceleration of intracellular APP degradation because the intracellular APP protein level in PBP-expressing cells is rapidly reduced relative to cells lacking PBP, and because the C-terminal fragments of APP are decreased by PBP expression along with the secretion of sAPPβ. Finally, the effect of PBP expression on the secretion of APP is reversed by an endoplasmic reticulum (ER) protease inhibitor. It is concluded that PBP plays a major role in APP metabolism.

Materials and Methods

Antibodies: Monoclonal antibody 22C11, which recognizes amino acid residues 66-81 of APP₆₉₅, was purchased from Roche (IN, USA). Anti-GID, a rabbit polyclonal antibody against amino acid residues 175-186 of APP₆₉₅, was provided by Dr. Greg Cole at University of California (CA, USA) and has been well characterized (Schubert et al. (1989) Neuron, 3: 689-694). CT-15, a rabbit polyclonal antibody against the last 15 C-terminal amino acid residues of APP₆₉₅ was provided by Dr. Edward Koo at University of California (San Diego, USA). Monoclonal antibody 6E10, which recognizes amino acid residues 1-17 of human Aβ, was purchased from Senetek (CA, USA). An affinity-purified polyclonal antibody which recognizes amino acid residues 2012-2027 of PBP was generated in rabbits (Kashiwa et al. (2000) J. Neurochem 75: 109-116). A polyclonal antibody, sc-6018, against a peptide mapping at the C-terminal of human laminin β-1 and a rat anti-presenilin monoclonal antibody, recognizing the N-terminal residues 21-80, were purchased from Chemicon (CA, USA).

Cells and transfection: Neuronal cell line B103 (Schubert et al. (1974) Nature, 249, 224-227) was grown in Dulbecco's modified Eagle's medium (DMEM) supplemented with 10% heat-inactivated fetal bovine serum (FBS). B103 cells were transfected with APP₆₉₅ using G418 selection (Schubert et al. (1989) Neuron, 3: 689-694) and with various plasmids by Lepfectamin2000 (GIBCO; NJ, USA) using puromycin selection for PBP or hygromycin for PS1, respectively. The stably transfected cells were subsequently cloned and screened for protein expression by western blot analysis.

Western blotting, metabolic labeling and immunoprecipitation: For western blotting, cells were washed twice with ice-cold PBS and lysed in lysis buffer (1% Triton, 50 mM Hepes pH 7.5, 50 mM NaCl, 5 mM EDTA, 1 mM Na₃VO₄, 50 mM NaF, 10 mM Na₄P₂O₇, plus a mixture of protease inhibitors (Complete Mini; Roche, Ind., USA). Protein concentrations were determined by Coomassie Plus (Pierce; IL, USA). The same amount of protein from each sample was separated on Novex pre-cast polyacrylamide gels (Invitrogen; CA, USA), and transferred to Immobulin membranes (Millipore; MA, USA). The membranes were blocked with 5% non-fat milk in Tris-buffered saline for 1 hour at room temperature. Following overnight incubation at 4° C. with primary antibodies, the antigens are detected with horseradish peroxidase-conjugated secondary antibodies (Bio-Rad; CA, USA; 1:20,000), an ECL kit (Amersham; NJ, USA), and exposed to films. For pulse-chase experiments, cells were grown to 80% confluency and incubated in methionine-free DMEM for 90 min. Cells were labeled with [³⁵]S-methionine (400 μCi/ml in methionine-free DMEM plus 5% dialyzed FBS) for 10 min at 37° C., and the medium was replaced with serum-free DMEM medium plus Ni-supplement (Sigma Inc.; MI, USA). The media were collected and cells were lysed after different time periods. Protein concentrations were determined and the same amounts of proteins were immunoprecipitated with antibodies at 4° C. overnight 25 μl of anti-mouse IgG agarose (Roche) was then added to each sample and incubated at 4° C. for 2 hours on a rocker platform. The immunoprecipitates were collected by centrifugation and washed 4 times with the washing buffer (0.1% Triton, 20 mM Hepes, 150 mM NaCl, 10% glycerol). The agarose beads were resuspended in 30 μl SDS-PAGE sample buffer boiled for 3 min to release the proteins. After 2 min of centrifugation, the supernatants were separated on 8% Tri-glycine gels. The gels were dried and subjected to autoradiography and quantitated by NIH image.

Total RNA Isolation and RT-PCR: Total RNAs were isolated from the cells and brain tissues using RNA STAT-60 kit (TEL-TEST “B”, Inc.; TX, USA). Reverse transcription (RT) reactions were performed for each RNA sample using 1 μg of total RNA in reverse transcription buffer composed of 10 mM DTT, 20 μM each of dATP, dCTP, dGTP, and dTTP, and 1 μM of oligo(dT)₁₅₋₁₈. The solution was heated to 65° C. for 5 min and cooled to 37° C. for 10 min, and then incubated in the presence of 25 units of AMV reverse transcriptase at 42° C. for 1 hour. Master mixes for PCR reaction were used for each sample. The PCR reaction mixture contained forward and reverse primers (10-20 pmol each), dNTPs (200 μM each as final concentration), lox PCR buffer, Taq DNA polymerase (0.5 units) (Roche; IN, USA), 1 μl of the RT mixture as the source of cDNA. The primers used for PCR reaction were as follows: 5′ATGGATGCAGAATTCCGACATGAC3′ (SEQ ID NO:13; forward) (nt1933-1956) and 5′CTAGTTCTGCATCTGCTCAAAGAA3′ (SEQ ID NO:14; reverse) (nt2235-2212) for APP gene (GeneBank Y00264). Amplification was performed at 94° C. for 40 sec, 56° C. for 1 min, and 72° C. for 1 min, for 35 cycles. The PCR reactions of water with primers and RNAs without RT reaction were also conducted as a control. After amplification, each sample was electrophorased on a 1.5% agarose gel visualized by ethidium bromide staining.

### RESULTS AND CONCLUSIONS

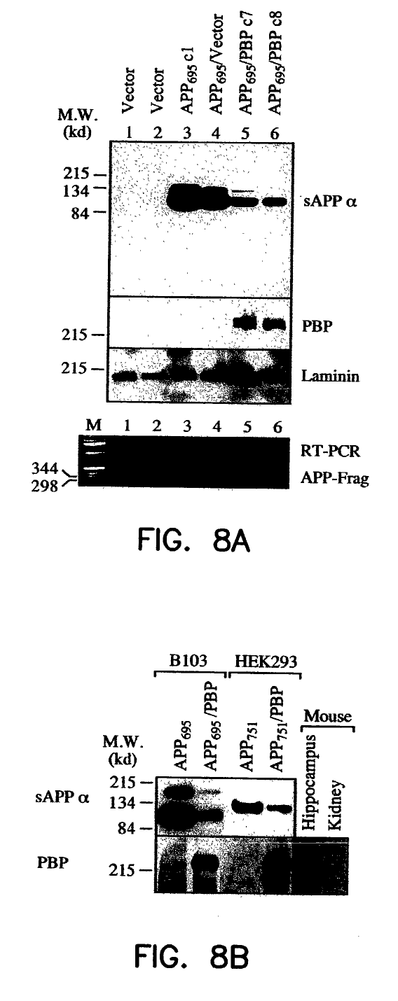

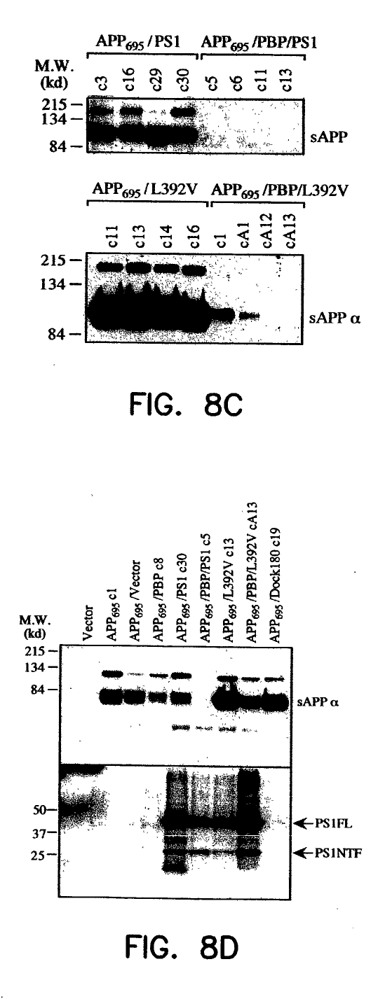

To investigate the effect of PBP on APP protein secretion, a rat nerve cell line called B103 (Schubert et al. (1974) Nature 249: 224-227) was transfected with APP₆₉₅, and again with a plasmid harboring the full length PBP cDNA. B103 cells normally express little or no APP and no PBP. The expression of the PBP protein was detected in transfected clones of B103 (APP₆₉₅/PBP) at a level similar to that expressed in the mouse hippocampus (FIGS. 8a and b). Secreted proteins from the stably transfected cells containing either an empty vector or the PBP gene were first studied by Western blotting using antibody 6E10, which recognizes amino acid residues 1-17 of human Aβ and sAPPα (Kim et al. (1990) Neurosci. Res. Comm., 7: 113-122). The secretion of sAPPα was dramatically decreased in all independently isolated clones expressing PBP relative to clones containing empty vector (FIG. 8a). To determine the specificity of the PBP effect on APP secretion and to eliminate the possibility of clonal variation, several control experiments were carried out. First, the effect of PBP expression on the secretion of another protein, β-laminin, was examined; no effect was observed (FIG. 8a). Second, a transcriptional change was ruled out because the abundance of APP mRNAs was not altered (FIG. 8a). Third, transient transfection of PBP into B103 (APP₆₉₅) cells led to the reduction of APP secretion in an uncloned population (FIG. 8b). Fourth, the secretion of APP was not affected by the overexpression of a protein which has a 40% homology with PBP (Kashiwa et al. (2000) J. Neurochem., 75, 109-116) (DOCK180, FIG. 8d). Lastly, the secretion of endogenous APP₇₅₁ was reduced in HEK293 cells expressing PBP (FIG. 8b). Therefore, the PBP protein is involved in APP protein metabolism.

Since in situ hybridization shows that PBP and PS1 are expressed in overlapping areas of the brain (Kashiwa et al. (2000) J. Neurochem. 75: 109-116), it was asked if the effect of PBP on APP secretion is affected by PS 1. Because B103 cells express very low levels of endogenous PS1, the fill-length PS1 gene was stably-transfected into the B103(APP₆₉₅/PBP) cells and the secreted sAPPα assayed. FIGS. 8c and d show that sAPPα secretion was further reduced relative to cells expressing PBP alone in four independently isolated clones which co-express PBP and wild-type (WT) PS1 or the mutant FAD PS1 (L392V).

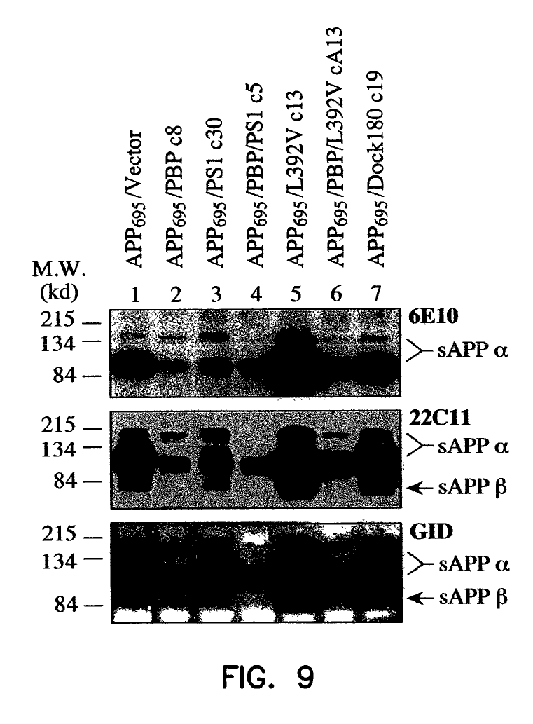

Two major pathways have been described for the cellular processing of APP (De Strooper et al. (2000) J. Cell Sci., 113: 1857-1870). The α-secretion pathway is mediated through the cleavage of APP within the Aβ sequence thereby excluding the generation of Aβ peptides, while the β-secretion pathway is responsible for the generation of sAPPβ and β-amyloid peptides. To determine if PBP also regulates the β-secretion pathway, antibodies 22C11 and GID, which recognize both sAPPα and sAAPβ (Schubert et al. (1989) Neuron, 3: 689-694; Weidemann et al (1989) Cell 57: 115-126), and antibody 6E10, which only recognizes sAPPα (Kim et al. (1990) Neurosci. Res. Comm, 7: 113-122), were used to examine the secreted forms of APP. Less secretion of sAPPβ was observed, as well as sAPPα in PBP-containing cells compared to cells without PBP (FIG. 9). The specific sAPPβ molecules are indicated by arrows.

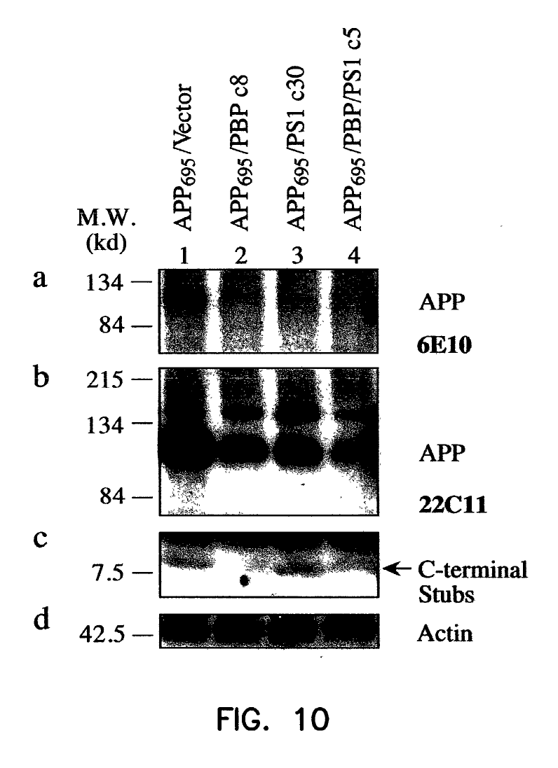

The decreased secretion of APP caused by PBP may be due to a blockage of the secretion pathway, which will cause an intracellular accumulation of APP, or to an increase in the rate of APP degradation. To distinguish between these alternatives, the intracellular levels of APP were examined. Full length APPs was reduced in B103 cells containing APP₆₉₅/PBP, APP₆₉₅/PS1, or APP₆₉₅/PBP/PS1, relative to APP₆₉₅ cells as defined by two antibodies, 6E10 and 22C11 (FIGS. 10a and b). In addition, the expression pattern of the C-terminal fragments of APP was similar to the secretion pattern of sAPP in the different cell types as determined by immunoprecipitation with the CT-15 antibody, which recognizes the last 15 C-terminal amino acid residues of APP₆₉₅ (FIG. 10c). These data rule out the possibility that PBP expression is enhancing the intracellular accumulation of APP by PBP and suggest that the decreased APP secretion caused by PBP expression is not due to a blockage in the secretion pathways.

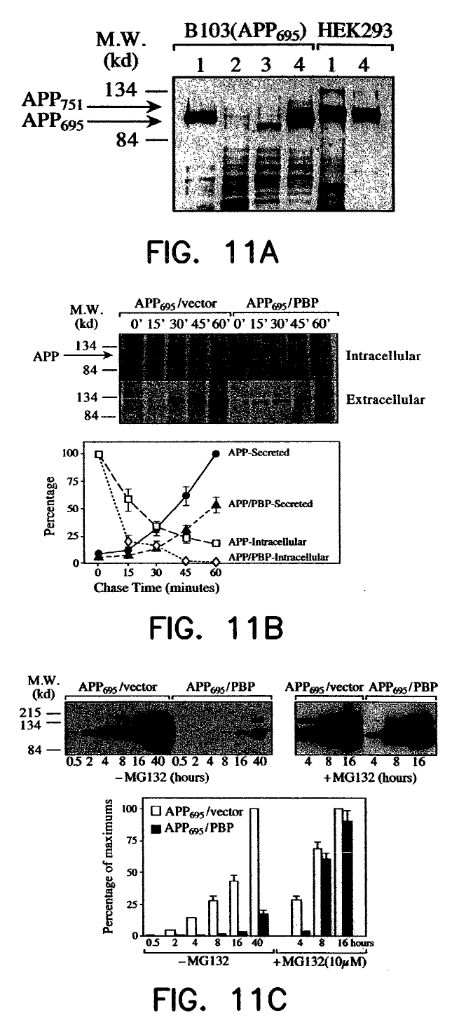

Since there is no increased intracellular accumulation of APP in cells expressing PBP, the effect of PBP on the turnover of APP was investigated by pulse-chase experiments. Antibodies 6E10 and CT-15 specifically immunoprecipitate the intracellular APP holoprotein, while antibodies GID and 22C11 performed less well (FIG. 11a). 6E10 was used in the following experiments. B103 cells expressing either APP₆₉₅/vector or APP₆₉₅/PBP were labeled for 10 min in ³⁵S-methionine-containing media and chased for 15, 30, 45, and 60 min in ‘cold’, serum-free conditioned media In control B103 (APP₆₉₅/vector) cells, the APP protein level was gradually reduced (FIG. 11b). In contrast, the intracellular APP protein level in B103 (APP₆₉₅/PBP) cells rapidly decreased. The initial levels following the 10 min labeling period were also lower than controls in cells expressing PBP, a result which would be predicted, for nascent proteins can be very rapidly degraded after synthesis (Schubert et al. (2000) Nature, 404: 770-774). There was a temporal increase in the amount of APP protein in the medium of both cell types (FIG. 11b). However, since the overall level of APP accumulation in the medium is decreased by the expression of PBP (FIG. 8), it is likely that PBP facilitates the degradation of APP.

To independently verify the data on APP degradation, the effect of two protease inhibitors, leupeptin and MG132, on the secretion and the intracellular accumulation of APP was studied. Leupeptin is thought to inhibit lysosomal proteases, whereas MG132 is thought to inhibit ER membrane protein degradation through the ubiquitin-proteasome pathway (Soriano et al. (1999) J. Biol. Chem., 274: 32295-32300). Both APP secretion and the intracellular accumulation of APP were significantly increased in the APP₆₉₅/PBP cells by 10 μM MG132; no effect was observed in either cells treated by 100 μM leupeptin (data not shown). More strikingly, the secretion of APP in the cells expressing PBP was enhanced in concert with increasing duration of MG132 treatment and was almost restored to the control level after 16 hours (FIG. 11c). These data again show that the APP degradation is affected by PBP; they also confirm the RT-PCR data showing no effect on APP transcription.

The data show that PBP decreases the secretion of APP and that this is due to the acceleration of intracellular APP degradation. This conclusion is based upon the following observations: 1) There is no accumulation of intracellular APP in cells expressing PBP. 2) The intracellular APP protein level is rapidly reduced in cells expressing physiological levels of PBP relative to cells not expressing PBP. 3) The level of sAPP secretion and the remaining C-terminal fragments of APP are decreased by PBP to a similar extent. 4) The secretion of sAPPβ is also decreased by PBP. 5) The ER protease inhibitor, MG132, increases the accumulation of intracellular APP and reverses the effect of PBP on APP secretion. If MG132 is ER specific (Soriano et al. (1999) J. Biol. Chem., 274: 32295-32300), then the pleotropic effects of PBP and PS1 on APP degradation may occur in the ER. These results are consistent with the cellular localization of both PS1 and PBP, for PS1 is found in the ER/Golgi (Doan et al. (1996) Neuron, 17:1023-1030; Xia et al. (1998) Biochemistry, 37: 16465-16471) and PBP redistributes from cytoplasm to intracellular membranes in the presence of PS (Kashiwa et al. (2000) J. Neurochem., 75: 109-116). Since both a-and P-secretion pathways are affected, PBP has a general effect on APP degradation and processing.

PS1 binds to a number of proteins (Van Gassen et al. (2000) Neurobiol. Dis. 7, 135-151) including several implicated in protein sorting, degradation and trafficking. The latter include G₀ (Smine et al. (1998) J. Biol. Chem., 273: 16281-16288), RabII (Dumanchin et al. (2000) Human Molec. Genet., 9:1263-1269), RabGDI (Scheper et al. (2000) Human Molec. Genet., 9: 303-310), the 170 kDa cytoplasmic linker protein (CLIP 170/Restin) (Johnsingh et al. (2000) FEBS Lett., 465: 53-58), and most recently nicastrin (Yu et al. (2000) Nature, 407: 48-54). PBP contains a SH₃-binding domain, interacts with the Crk adaptor protein, and shares a 40% homology with DOCK180 (Kashiwa et al. (2000) J. Neurochem., 75: 109-116). Since DOCK180 interacts with Rae and other small G-proteins, PBP may also interact with small G-proteins involved in protein breakdown, or mediate phosphorylation events between proteins involved in APP metabolism. It is becoming clear that PS1 is part of a multiprotein complex which controls several aspects of cellular physiology, including APP metabolism (Sisodia (2000) Science, 289: 2296-2297) and protein breakdown (Niwa et al. (1999) Cell, 99: 691-702; Katayama et al. (2000) Nature Cell Biol., 1: 479-485). Since PBP functionally interacts with PS1 to alter APP metabolism, it is likely that PBP is another member of the complex. Due to its size, PBP may indeed be its functional scaffold.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially pure Presenilin/Crk binding polypeptide, wherein the polypeptide is characterized by:
(a) interacting with a presenilin polypeptide;
(b) modulating intracellular calcium mobilization; and
(c) redistributing from cytoplasm to membrane in the presence of a presenilin polypeptide
and conservative variants thereof.

2. A polypeptide of claim 1, wherein the polypeptide is characterized by:
(a) interacting with a Crk polypeptide; and
(b) containing at least one SH3 domain.

3. A polypeptide of claim 1, wherein the polypeptide has an expression pattern in the brain.

4. A polypeptide of claim 1, wherein the polypeptide is a mammalian polypeptide, preferably a human polypeptide.

5. A polypeptide of claim 1, wherein the polypeptide has at least about 60% homology, preferably at least about 70% homology, to the amino acid sequence set forth in SEQ ID NO:2.

6. Biologically active fragments of a polypeptide of claim 1.

7. The polypeptide of claim 1, wherein the polypeptide has a sequence substantially the same or the same as the amino acid sequence set forth in SEQ ID NO:2.

8. A polypeptide of claim 1, wherein the polypeptide is encoded by SEQ ID NO:1 or degenerative variants thereof.

9. A polypeptide of claim 1, wherein the modulating of intracellular calcium is a decrease or increase in intracellular calcium.

10. A polypeptide of claim 1, wherein the polypeptide comprises an amino acid sequence containing residues PPXLPXKR (SEQ ID NO:4).

11. A substantially pure or isolated peptide having the amino acid sequence PPXLPXKR (SEQ ID NO:4).

12. An antibody that binds to a polypeptide of claim 1 or to immunoreactive fragments thereof.

13. A substantially pure peptide having the amino acid sequence EERGVKLGLGDFIFYSVLVGKA (SEQ ID NO:3).

14. An isolated polynucleotide encoding a polypeptide as in claim 1 or claim 2.

15. An isolated polynucleotide selected from the group consisting of:
a) a polynucleotide encoding a polypeptide having an amino acid sequence as set forth in SEQ ID NO:2;
b) a polynucleotide of a), wherein T can be U;
c) a polynucleotide complementary to a) or b);
d) a polynucleotide having a nucleotide sequence as set forth in SEQ ID NO:1;
e) degenerate variants of a), b), c) or d); and
f) a fragment of a), b), c), d) or e) having at least 15 base pairs and that hybridizes to a polynucleotide encoding a polypeptide as set forth in SEQ ID NO:2.

16. An isolated polynucleotide, wherein the nucleotide is at least 15 bases in length and will hybridize under moderate to highly stringent conditions to DNA encoding a polypeptide of SEQ ID NO:2.

17. An expression vector, wherein the expression vector comprises a polynucleotide of claim 16.

18. A host cell comprising a vector of claim 17.

19. A method for producing a polypeptide comprising the steps of:
(a) culturing a host cell of claim 18 under conditions suitable for the expression of the polypeptide; and
(b) recovering the polypeptide from the host cell culture.

20. A method for diagnosing a subject having or at risk of having a neurodegenerative disorder comprising detecting PCBP in a soluble and an insoluble fraction of CNS tissue from the subject, wherein a level of PCBP that is higher in the insoluble fraction as compared to the soluble fraction is indicative of a neurodegenerative disorder.

21. The method of claim 20, further comprising comparing the level of PCBP in a soluble and an insoluble fraction of CNS tissue from a control subject not having a neurodegenerative disorder with the sample from the subject suspected of having or at risk of having the disorder.

22. A method for diagnosing a subject having or at risk of having a neurodegenerative disorder associated with PCBP comprising detecting PCBP level or activity in a sample CNS tissue from the subject, wherein a difference in the level or activity of PCBP in the subject as compared to a normal subject is indicative of a neurodegenerative disorder.

23. A method for identifying a compound that modulates intracellular calcium mobilization comprising:
(a) incubating the compound with a cell expressing a Presenilin/Crk binding polypeptide or fragment thereof and a presenilin and/or Crk polypeptide under conditions sufficient to permit the compound to interact with the cell; and
(b) comparing intracellular calcium mobilization in a cell incubated with the compound with the intracellular calcium mobilization in a cell not incubated with the compound, wherein a difference in calcium mobilization is indicative of a compound that modulates intracellular calcium mobilization.

24. The method of claim 23, wherein the compound is selected from peptides, peptidomimetics, polypeptides, pharmaceuticals, chemical compounds, biological agents, antibodies, neurotropic agents and anti-epileptic agents.

25. A method of treating a disorder associated with intracellular calcium mobilization comprising administering to a subject in need thereof a therapeutically effective amount of a compound that modulates Presenilin/Crk binding polypeptide activity or level, thereby treating the disorder.

26. A method for identifying a compound that modulates Presenilin/Crk binding polypeptide (PCBP) activity or expression comprising:
(a) incubating components comprising the compound and PBCP or a polynucleotide encoding PCBP and presenilin and/or Crk under conditions sufficient to permit the components to interact; and
(b) determining the effect of the compound on PCBP activity or expression.

27. A kit useful for the diagnosis of a subject having or at risk of having a neurodegenerative disorder comprising carrier means comprising at least one container containing a reagent that interacts with a Presenilin/Crk binding polypeptide thereby permitting the level of Presenilin/Crk binding polypeptide to be determined.

28. A method of treating a subject having or at risk of having a disorder associated with a decreased level or activity of a Presenilin/Crk binding polypeptide, as compared to a subject not having the disorder comprising:
introducing into a subject having or at risk of having the disorder a polynucleotide encoding the Presenilin/Crk binding polypeptide operatively linked to a regulatory sequence, thereby treating the subject.

29. A transgenic non-human animal whose genome comprises a disruption of a Presenilin/Crk binding polypeptide gene, wherein said disruption results in the animal exhibiting a disorder of the nervous system as compared to a wild-type animal not having the disruption.

30. The transgenic knockout animal of claim 29, wherein the animal is homozygous or heterozygous for said disruption of the endogenous Presenilin/Crk binding polypeptide gene.

31. A method for producing a transgenic mouse exhibiting a disorder of the nervous system, said method comprising:
(a) introducing a transgene comprising a selectable marker sequence into a mouse embryonic stem cell;
(b) introducing the mouse embryonic stem cell into a mouse embryo;
(c) transplanting the embryo into a pseudopregnant mouse;
(d) allowing the embryo to develop to term; and
(e) identifying a transgenic mouse whose genome comprises a disruption of the endogenous Presenilin/Crk binding polypeptide gene, wherein the disruption results in the mouse exhibiting a disorder of the nervous system as compared to a wild-type mouse.

32. A method of regulating the level of β-amyloid precursor protein in vitro or in vivo, as compared to an untreated cell or subject, comprising:
introducing into a subject or a cell a sense or antisense polynucleotide encoding the Presenilin/Crk binding polypeptide operatively linked to a regulatory sequence.
